(19) United States
(12) Patent Application Publication
(10) Pub. No.: US 2023/0381393 A1
(43) Pub. Date: Nov. 30, 2023

(54) DEVICES AND METHODS FOR RECOVERING DISEASE-CAUSING TOXIC CONSTITUENTS IN THE BLOOD

(71) Applicant: ACTORIUS INNOVATIONS RESEARCH CO, Simi Valley, CA (US)

(72) Inventors: Jayant Jagannath KHANDARE, Pune (IN); Gourishankar Rajshekhar Aland, Pune (IN)

(21) Appl. No.: 17/817,934

(22) Filed: Aug. 5, 2022

(30) Foreign Application Priority Data
May 27, 2022 (IN) 202241030495

(51) Int. Cl. A61M 1/36

(52) U.S. Cl. CPC A61M 1/362; A61M 1/3615

(57) ABSTRACT

The present disclosure relates to non-hemolytic blood compatible devices and methods for capture, enumeration, removal of disease-causing agents from the blood and for the treatment of the cancer patients. The said devices incorporating non-hemolytic compositions are useful for removing disease-causing agents ‘ex vivo’ from cancer patient's blood to prevent/delay the proliferation of cancer. The devices retain disease-causing agents in particular Circulating Tumor Cells (CTCs), allowing the passage of other blood constituents retaining the viability of hematopoietic cells.

### CROSS-REFERENCE TO RELATED APPLICATIONS

This application claims the benefit of priority under 35 U.S.C. § 119 to Indian Patent Application Serial No. 202241030495, filed on 27 May 2022, which is hereby incorporated herein by reference in its entirety.

### FIELD OF INVENTION

The present disclosure relates to devices for removing disease-causing agents from the blood of cancer patients. It relates to devices comprising non-hemolytic compositions for the removal of disease-causing agents ‘ex vivo’ for the prevention as well as monitoring the progression of the disease. The devices adsorb the CTCs and other toxic constituents from the blood of a patient. Methods of capture, enumeration, removal of the said disease-causing agents, as well as the treatment of the cancer patient are disclosed.

### BACKGROUND

Advances in anti-cancer therapeutics are being focused on personalized treatments, using newer chemical and biological entities that can target multiple cellular and immune pathways. The anti-cancer therapies predominantly include conventional chemotherapy, immunomodulatory therapies (e.g., anti-CTLA-4, anti-PD1/PDL1), or combinatorial therapy. Increasing clinical evidence shows that cancer is a systemic disease with localized presentations. Subsequently, anti-cancer therapeutics over the past decades have, with varying degrees of success, been targeted towards primary tumorigenesis. However, primary tumorigenesis accounts for only 10% of cancer mortality. 90% of cancer-related deaths are attributed to the spread of cancer to distant organs, known as metastasis (Singh B. et al. J. Mater. Chem. B. 2021; 9, 2946-2978). Thus, early metastasis detection and the treatment thereof remain the primary objective in improving the progression-free survival (PFS) and overall survival (OS) in these patients.

Unfortunately, the development of therapies aimed at preventing metastatic disease has proved challenging and represents a major unmet medical need in cancer management. The early detection of occult-metastases or Minimal Residual Disease (MRD) is highly challenging and the failure to do so is implicated in the potential of relapse and remission, further highlighting the need to target metastatic cancers using novel therapeutic approaches. (Pantel K., Alix-Panabières C. Nat Rev Clin Oncol. 2019; 16, 409-424).

Metastasis has been directly correlated with the presence of circulating tumor cells (CTCs) in the blood of a cancer patient. CTCs are the result of solid cancerous lesions that are continuously shed from a primary tumor site and disseminate systemically via peripheral blood circulation eventually reaching the distant organs to establish a metastatic niche.

CTCs play a dominant role in tumor invasion and extravasation but also carry vital prognostic information regarding tumor onset, progression, and metastasis (Habli, Z. et al. Cancers (Basel). 2020; 12, 1930). In over 400 clinical studies, CTCs have been identified as independent risk factors in numerous carcinomas and clinically validated as active and aggressive biomarkers of metastasis. These clinical studies have correlated CTC presence directly with progression-free survival (PFS), disease-free survival (DFS), and overall survival (OS). (Cristofanilli, M. et al. N Engl J Med. 2004; 351, 781-791; De Bono J. S. et al. Clin. Cancer Res., 2008, 14, 6302-6309; Botteri E. et al. Breast Cancer Res. Treat. 2010; 122, 211-217; Cohen S. J. et al. J. Clin. Oncol. 2008; 26, 3213-3221; Hayes D. F. et al. Clin. Cancer Res. 2006; 12, 4218-4224; Miller M. C. et al. J. Oncol. 2009; 2010, 617421). CTCs possess specific phenotype signatures unique from other normal epithelial cells and hence are employed in cancer diagnostics, real-time monitoring of the treatment responses, assessing cancer relapse and remission. However, the low abundance of CTCs, in the range of 1-10, as compared to billions of blood cells per milliliter of peripheral blood of metastatic cancer patients makes CTC isolation and removal extremely challenging. (Allard W. J. et al. Clin. Cancer Res. 2004; 10, 6897-6904).

Conventional anti-cancer therapies (e.g., neoadjuvant chemotherapy (NACT), adjuvant chemotherapy (AT), immunotherapy, etc.), while successful against primary solid tumors, have little or no effect on CTCs in blood circulation and consequently are ineffective in controlling metastasis. It is recognized that metastasis detection, which involves CTC detection, enumeration, and accounting of the phenotype and genotype is important for the treatment decisions. The presence of one or more CTC predicts early recurrence and decreased overall survival in chemo naïve patients with non-metastatic breast cancer. (Lucci A. et al, Lancet Oncol. 2012; 13, 688-695).

In addition to diagnostic and prognostic value, removal of CTCs from cancer patient's blood would result in the reduction of extravasation and invasion process of the disease, thereby increasing the overall survival of a patient (U.S. patent application Ser. No. 13/077,427; U.S. patent application Ser. No. 14/452,828). Furthermore, the removal of CTCs as a downstream process has immense applications in both cellular and mutation analysis, including in drug discovery and development.

There is evidence in animal metastasis models, indicating that the removal of CTCs leads to an increased survival rate in the diseased host. Studies have indicated that even one time removal of CTCs in mouse models with induced human ovarian cancer has led to increased survival and slower tumor progression. Significantly, a mouse tumor generated from cultured CTCs was twice as large as a regular tumor after 3 weeks (Ameri K. et al., Br J Cancer. 2010; 102, 561-569; Scarberry K. et al. Nanomedicine (Lond.). 2011; 6, 69-78). One study suggests that CTCs attract WBCs and platelets to form metastasizing niches, and removal of CTCs inhibits the metastatic process. (Labelle M. et al, PNAS. 2014; 111, E3053-E3061). Furthermore, in mice with breast cancer, sustained CTC removal over a prolonged time period was correlated with reduced metastatic lesions in the lungs and liver. Removing CTCs even once from the mice with ovarian cancer resulted in a 10-fold decrease in tumor progression and 32% increase in overall survival (Azarin S. M. et al. Nat Commun. 2015; 6, 8094).

Such convincing pre-clinical evidence supports the necessity to develop methods, techniques, and devices, both implantable and extra-corporeal, for the removal of CTCs from the blood of the cancer patients.

### SUMMARY

The present disclosure is related to methods and devices utilized in the capture and removal of specific cells/targets and/or toxic constituents in blood or other bodily fluids for determination, monitoring and treatment. In a particular aspect, disclosure provides methods and devices for the removal of cancer cells/targets and/or toxic constituents in blood or other bodily fluids for determination, monitoring and treatment of cancer patients. In particular, the disclosure provides non-hemolytic adsorbent compositions in an extracorporeal device which removes the toxic constituents from the blood of cancer patients, and methods of treatment of a cancer patient using the device.

In an aspect, the disclosure provides a hemocompatible device for the capture, isolation, identification, and/or removal of a disease-causing agent from a biological sample of a subject or patient, e.g., a fluid such as blood, serum, spinal fluid, urine, etc. In any aspect or embodiment described herein, the device comprises a channel and an adsorbent that selectively binds to the disease-causing agent, thereby allowing the passage of the blood through the device preserving the morphology and viability of the red blood cells.

In any aspect or embodiment described herein, the device for the capture and removal of the disease-causing agent comprises a hollow column or channel. In any aspect or embodiment described herein, the device comprises a hollow column or channel, and a reservoir. In any aspect or embodiment described herein, the device comprises a spiral or coiled hollow column or channel.

In any aspect or embodiment described herein, the device comprises multiple hollow columns. In any aspect or embodiment described herein, the device comprises multiple hollow columns arranged in series. In any aspect or embodiment described herein, the device comprises multiple hollow columns arranged in parallel.

In any aspect or embodiment described herein, the device is fabricated from a material selected from glass, steel, silicone, fluorinated polymers.

In any aspect or embodiment described herein, the device comprises an internal surface or internal volume that includes an adsorbent. In certain embodiments, the internal surface is functionalized and linked or coupled to a ligand that selectively binds to the disease-causing agent, wherein the disease-causing agent is a circulating cancer cell. In certain embodiments, the internal volume comprises an adsorbent that is functionalized and linked or coupled to a ligand that selectively binds to the disease-causing agent, wherein the disease-causing agent is a circulating cancer cell. In any aspect or embodiment described herein, the device comprises more than one ligand and/or more than one type of ligand that selectively binds to the circulating cancer cells (CTCs). In any aspect or embodiment described herein, the sensitivity and specificity for the capture of CTCs is enhanced. In any aspect or embodiment described herein, the device captures MCF7 cells, A549 cells and CTCs.

In any aspect or embodiment described herein, the capture of CTCs from the biological sample is due to specific binding to CTC markers, such as epithelial cell adhesion molecule (EpCAM), human epidermal growth factor receptor-2 (HER-2), epidermal growth factor receptor (EGFR), carcinoembryonic antigen (CEA), prostate specific antigen (PSA), CD24, and folate binding receptor (FAR).

In any aspect or embodiment described herein, the flow of the biological samples, e.g., fluid such as blood, serum, spinal fluid urine, etc. is in the laminar region.

In any aspect or embodiment described herein, the adsorbent substrate is glass beads. In any aspect or embodiment described herein, the adsorbent is glass beads that are functionalized and linked to a ligand that selectively binds to the circulating cancer cells. In any aspect or embodiment described herein, the adsorbent glass beads are packed into the device, and the packed volume is in the range 10% to 90% v/v, including all values and ranges in between.

The hemocompatible device of claim 1, wherein the disease-causing agent is a circulating tumor cell selected from a breast cancer cell, a prostate cancer cell, a colorectal cancer cell, a lung cancer cell, a pancreatic cancer cell, an ovarian cancer cell, a bladder cancer cell, an endometrin or uterine cancer cell, a cervical cancer cell, a liver cancer cell, a renal cancer cell, a thyroid cancer cell, a bone cancer cell, a lymphoma cell, a melanoma cell and a non-melanoma skin cancer cell.

In additional aspects, the disclosure provides methods for the capture, isolation, and/or removal of a disease-causing agent from a biological sample of a cancer patient, e.g., a fluid such as blood, serum, spinal fluid, urine, etc. In any of the aspects or embodiments described herein, the method includes providing a device as described herein, and allowing the biological sample to flow through the device thereby capturing, isolating and/or removing the disease-causing agent, wherein the method is effective for the capture, isolation and/or removal of the disease-causing agent from the biological sample of the cancer patient. In any of the aspects or embodiments described herein, the method includes an additional step of identifying the disease-causing agent.

In any of the aspects or embodiments described herein, the biological sample, e.g., fluid such as blood, serum, spinal fluid, urine, etc. is passed through the device over a period of from about 5 minutes to about 60 minutes.

In any of the aspects or embodiments described herein, the disease-causing agent is a circulating tumor cell selected from a breast cancer cell, a prostate cancer cell, a colorectal cancer cell, a lung cancer cell, a pancreatic cancer cell, an ovarian cancer cell, a bladder cancer cell, an endometrin or uterine cancer cell, a cervical cancer cell, a liver cancer cell, a renal cancer cell, a thyroid cancer cell, a bone cancer cell, a lymphoma cell, a melanoma cell and a non-melanoma skin cancer cell.

In any of the aspects or embodiments as described herein, the method includes the step of treating the captured or isolated cancer cell with an anticancer drug, e.g., Vancomycin, Metformin, Doxorubicin, Methotrexate, Paclitaxel, 5-Fluorouracil, Cisplatin, and Camptothecin, Docetaxel, Oxaliplatin, and Cyclophosphamide, wherein the anticancer drug inhibits the growth, differentiation or induces apoptosis of the cancer cell.

In additional aspects, the disclosure provides methods for the capture, isolation, and destruction of a disease-causing agent from a biological sample of a cancer patient, e.g., a fluid such as blood, serum, spinal fluid, urine, etc. comprising the steps of providing a device as described herein, and allowing the biological sample to flow through the device thereby capturing or isolating the disease-causing agent, wherein the disease-causing agent is a cancer cell, and then treating the captured or isolated cancer cell with an anti-cancer drug. In any of the aspects or embodiments as described herein, anticancer drug is selected from, e.g., Vancomycin, Metformin, Doxorubicin, Methotrexate, Paclitaxel, 5-Fluorouracil, Cisplatin, and Camptothecin, Docetaxel, Oxaliplatin, and Cyclophosphamide, wherein the anticancer drug inhibits the growth, differentiation or induces apoptosis of the cancer cell.

In additional aspects, the disclosure provides methods of treating a cancer patient, comprising the steps of a) removing an amount (e.g., a fixed amount) of a biological sample, e.g., a fluid such as blood, serum, spinal fluid, urine, etc. from the cancer patient, b) passing the blood through a hemocompatible device as described herein for capturing or isolating a cancer cell, c) detecting the cancer cells present in the biological sample, captured or isolated after the passage of the biological sample through the hemocompatible device and measuring the number of cancer cells in the biological sample, and e) and infusing the treated blood back in to the cancer patient, wherein the method is effective for removing cancer cells from the blood of the patient but preserves the morphology and viability of the cancer patient's red blood cells.

In any aspect or embodiment described herein, the method includes prior to step (b), the step of estimating the number of cancer cells present in the amount of biological sample, e.g., a fluid such as blood, serum, spinal fluid, urine, etc. withdrawn by measuring the number of cancer cells in a test sample, e.g., in a sample of 1 to 1.5 milliliters.

In any aspect or embodiment described herein, the method includes prior to step (c), the step of estimating the number of the cancer cells present in the amount of biological sample, e.g., a fluid such as blood, serum, spinal fluid, urine, etc. recovered after the passage of the biological sample through the hemocompatible device by measuring the number of cancer cells in a sample of 1 to 1.5 milliliters.

In any aspect or embodiment described herein, the biological sample is blood. In any of the aspects or embodiments, the disease-causing agent or cancer cell is a circulating tumor cell.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the present disclosure may be utilized in numerous combinations, all of which are expressly contemplated by the present description. These additional advantages, objects and embodiments are expressly included within the scope of the present disclosure. The publications and other materials used herein to illuminate the background of the disclosure, and in particular cases, to provide additional details respecting the practice, are incorporated by reference in their entirety for all purposes.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating an embodiment of the disclosure and are not to be construed as limiting the invention. Further objects, features and advantages of the invention will become apparent from the following detailed description taken in conjunction with the accompanying figures showing illustrative embodiments of the present disclosure, in which:

FIG. 1. A spiral channel device showing separate top plate A and bottom plate B.

FIG. 2. A spiral channel device showing top plate A and bottom plate B overlayed together.

FIGS. 3A, 3B, 3C, and 3D. Device having a circular spiral channel flow path. (3A) top part of the device, (3B) bottom part of the device, (3C) and (3D) oval shape designs, top part and bottom part.

FIGS. 4A, 4B, 4C, and 4D. Cross sectional view of circular spiral channel device showing different locking, leak proof mechanism designs of top and bottom plates.

FIG. 5. Circular spiral channel device filled with adsorbent glass beads, enclosed in a casing.

FIG. 6. Two circular spiral channel devices filled with adsorbent glass beads, each enclosed in a casing, in series.

FIGS. 7A and 7B. Spiral channel device: (7A) drawing view, (A-A), section view and (7B) detailed view.

FIG. 8. Computational fluid dynamic (CFD) analysis of spiral channel device filled with glass beads. Colormap image indicates pressure and velocity variation of fluid along with spiral flow channels.

FIG. 9. Variation of total CTCs captured and Hemolysis Index (HI) using glass beads of example 12 placed in spiral channel device (FIG. 5).

FIG. 10. Velocity measure of blood flow through spiral channel from 1 second to 20 seconds.

FIG. 11. A hollow cylindrical glass column arranged horizontally.

FIG. 12. Cylindrical glass columns in series arranged horizontally.

FIG. 13. Cylindrical glass columns arranged in parallel connected using medical grade silicone tubing.

FIG. 14. A cylindrical glass column incorporating the reservoir arranged horizontally.

FIG. 15. Cylindrical glass columns incorporating the reservoir in series arranged horizontally.

FIGS. 16A, 16B, and 16C. Different shapes of glass columns (i-iii) arranged horizontally.

FIG. 17. A hollow coiled glass column arranged horizontally.

FIG. 18. Hollow coiled glass columns in series arranged horizontally.

FIG. 19. Spiral silicone column arranged horizontally.

FIG. 20. Two spiral silicone columns arranged in series horizontally.

FIGS. 21A, 21B, 21C, 21D, and 21E. Cylindrical channel device comprising cylindrical body with 4 parallel spiral paths. (21A) Complete design, (21B) Cross-sectional view depicting press-fit connection details between the two device sections, (21C) front view of the device, (21D) top view of the device, (21E). left section of the device with press-fit connection extrusion.

FIG. 22. A hollow cylindrical glass column functionalized with (3-glycidyl-oxypropyl)-trimethoxysilane arranged horizontally.

FIGS. 23A, and 23B. (23A) A Hollow cylindrical glass column showing different packing volumes of glass beads of example 12. (23B) A hollow cylindrical silicone column sowing different packing volumes of glass beads of example 12.

FIG. 24. A hollow glass column with reservoir, packed with glass beads of example 12.

FIG. 25. A hollow U-shaped glass column packed with glass beads of example 12.

FIG. 26. A hollow spiral glass column packed with glass beads of example 12.

FIG. 27. A hollow spiral silicone column, packed with glass beads of example 12.

FIG. 28. Cancer patient red blood cell incubation on the spiral channel device (Example 1) for measurement of red blood cell hemolysis (RBC). White arrows indicate direction of blood flow.

FIG. 29. Brightfield microscopic analysis of the effect of substrates and intermediates non-hemolytic constituents on red blood cell architecture. Negative Hemolysis Control, Phosphate Buffered Saline pH 7.4. 2. Positive hemolysis Control 0.5% Triton-X 100. No changes observed in PBS treated cells. Triton-X treated cells indicate altered red blood cell architecture. Substrates and intermediates utilized in the synthesis of adsorbents as per example 11 from U.S. patent application Ser. No. 17/069,277 showing degradation of cells. Glass beads of example 11 from U.S. patent application Ser. No. 17/069,277 showed no change in RBCs.

FIG. 30. MCF7 cells captured on glass beads of example 12 placed inside spiral channel device (See FIG. 5). Inset shows magnified view of cells captured.

FIG. 31. Cluster of MCF7 cells captured on surface functionalized glass beads of example 12 placed inside the channel device (See FIG. 5). Inset shows magnified view of cells captured.

FIG. 32. A549 cells captured on glass beads of example 12 placed inside spiral channel device (See FIG. 5). Inset shows magnified view of cells captured.

FIG. 33. MCF7 cell detached using 1× trypsin from glass beads of example 12 incubated on spiral channel device (See FIG. 6).

FIG. 34. A549 cell detached using 1× trypsin from glass beads of example 12 placed inside the channel device (See FIG. 5).

FIG. 35. Cancer patient whole blood incubation with glass beads of example 12 placed inside the channel device (FIG. 5).

FIG. 36. Circulating tumor cell capture from cancer patient blood sample from glass beads of example 12 placed inside the spiral channel device (FIG. 5).

FIG. 37. MCF7 cells detached from glass beads of example 12 placed in spiral channel device.

FIG. 38. MCF7 cells detached from glass beads of example 30 from U.S. patent application Ser. No. 17/069,277 placed in spiral channel device (FIG. 5). (Effect of increase in incubation time).

FIG. 39. MCF7 cells detached from Tf conjugated glass beads of example 30 from U.S. patent application Ser. No. 17/069,277 placed in spiral channel device (FIG. 5). (Effect of increasing number of beads).

FIG. 40. MCF7 cells detached from glass beads of example 12 placed in spiral channel device (FIG. 5).

FIG. 41. MCF-7 cell capture from spiral channel device (FIG. 5) consisting glass beads of example 12 after incubation with different numbers of MCF-7 cells.

FIG. 42. Human breast cancer (MCF-7) cells capture with device with glass beads of example 12a) incubation of 300 cells per 580 mm² of cylindrical column device from example 26, b) incubation of 200 cells per 580 mm² of cylindrical column device, c) incubation of 100 cells per 580 mm².

FIG. 43. MCF-7 cell captured using spiral channel device (FIG. 5) with glass beads of example 12 after incubation with different number of MCF-7 cells.

FIG. 44. MCF-7 cell capture using spiral channel device (FIG. 5) linked with different antibodies containing glass beads of example 12, linked with different concentration of anti-epithelial cell adhesion molecule antibody, after incubation with fixed number of MCF-7 cells.

FIG. 45. Circulating tumor cell capture from spiral channel device (FIG. 5) using cancer patient blood with glass beads of example 12.

FIG. 46. Circulating tumor cell capture from cancer patient blood using spiral channel device (FIG. 5) with glass beads of example 12.

FIG. 47. Circulating tumor cell capture from cancer patient blood with spiral channel device (FIG. 5) containing glass beads of example 12 (packing volume 10%).

FIG. 48. Animal whole organs and tissue histopathology.

### DETAILED DESCRIPTION

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

Presently described are devices and methods of using the same for the capture or isolation of disease-causing agents, e.g., specific cells/targets and/or toxic constituents in blood or other bodily fluids for one or more of removal, destruction, determination, monitoring, measurement, and/or treatment. In a particular aspect, the disclosure provides methods and devices for the removal of cancer cells/targets and/or toxic constituents in blood or other bodily fluids for determination, monitoring and treatment of cancer patients. In particular, the disclosure provides non-hemolytic adsorbent compositions in an extracorporeal device which removes the toxic constituents from the blood of cancer patients, and methods of treatment of a cancer patient using the device.

Where a range of values is provided, it is understood that unless the context clearly dictates otherwise, each intervening value, to the tenth of the unit of the lower limit between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention unless the context indicates otherwise.

The following terms are used to describe the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting of the invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Adenocarcinoma such as lung, breast, colon, rectal, bladder, head and neck are organs of epithelial origin cancers. Cancer treatments are often incomplete due to the drug intolerance, non-specificity and targetability, acquired drug resistance, and severe side effects. Further, primary tumors can lead to metastasis through the process of extravasation and invasion mediated by circulating tumor cells (CTCs).

The presence of a tumor is confirmed by imaging the solid tissues using Computed Tomography/Positron Emission Tomography (CT/PET) and MRI. It is followed by solid tissue biopsy and histopathological staining. These methods are often limited due to invasiveness and inaccessibility of the tumour's specimen. Also, the sensitivity of CT/PET and MRI is such that the tumours of size less than 5 mm cannot be imaged or resolved. Hence the patient is considered to have no residual disease burden if the above-mentioned methods show an absence of tumor, before or even after the treatment.

It is well-established that at the origin of primary cancer, the tissue sheds the CTCs. CTCs because of their plasticity enter into peripheral blood circulation which further leads to clinical manifestations. Furthermore, these cells disseminate and circulate and colonize to form distant metastasis.

CTCs when in the form of group as ‘cell clusters’ are known to be up to 50 times more aggressive in translating the distant tumor metastasis compared to CTCs alone.

The presence of CTCs in blood circulation predicts and is associated with the progression of disease, short survival, failure to respond to treatment, and can aid in real-time monitoring of the patient for minimal residual disease. However, it is practically impossible to target CTCs and their destruction in blood circulation with sub-cellular cytotoxic drug concentrations and by any other therapy including radiation.

CTCs and other biomarkers such as cell-free nucleic acids (CfDNA, CtDNA), mutations like Epidermal growth factor receptor (EGFR), BReast CAncer gene (BRCA), and prostate serum antigen (PSA) can be used along with other tools, such as CT/PET imaging, biopsy, histopathological staining, mammography.

Thus, it is desirable in the art of cancer therapy to eliminate CTCs from patient's whole blood to reduce and eventually prevent metastatic progression and increase the patient's overall survival (Pantel, K., et al. Nat Rev Clin Oncol. 2019, 16, 409-424; Scarberry, K. E. et al. Nanomedicine. 2011, 6 1, 69-78; Azarin S., et al. Nat Comm 2015, 6, 8094). (Cohen et al. J. Clin. Oncol. 2008, 26, 3213-3221).

Similarly, the removal of other cancer-causing entities, for example, cell-free nucleic acids (CfDNA), cancer cells associated with nucleic acids (CtDNA), exosomes, and chemical entities such as chemo drugs would lead to increase the progression-free survival and overall survival of cancer patients.

Indeed, the identification and characterization of CTCs for cancer phenotype, genotype, and the organ of origin will lead to design of drugs which are better targeted. Also, the development of medical devices constituting non-hemolytic adsorbent compositions will be beneficial for cancer treatment for both early as well late-stage cancers.

It has now been surprisingly found that disease-causing agents, e.g., cancer causing agents such as cancer cells and CTCs, can be removed from the blood of a cancer patient using an extra-corporeal device containing non-hemolytic entities which can selectively adsorb the disease-causing agents.

Devices

In certain aspects, the disclosure provides a hemocompatible device for at least one of the capture, isolation, identification, enumeration, measurement, detection, removal and/or destruction of a disease-causing agent, for example, a cell such as a cancer cell including a circulating tumor cell, from a biological sample of a subject or patient, e.g., a fluid such as blood, serum, spinal fluid, urine, etc. In any of the aspects or embodiments described herein, the device is configured for the capture and removal of a disease-causing agent, for example, a cell such as a cancer cell including a circulating tumor cell, from a biological sample of a subject or patient, e.g., a fluid such as blood, serum, spinal fluid, urine, etc.

In any aspect or embodiment described herein, the device comprises a hollow channel extending along a longitudinal axis through the device. For example, in certain embodiments the device comprises a body defining an exterior surface and at least one hollow channel extending from a first opening on one end of the device to another opening on the opposite end of the device. In any aspect or embodiment, the hollow channel includes an adsorbent that selectively binds to the disease-causing agent, thereby allowing the passage or flow of the biological sample, e.g., a fluid such as blood, serum, spinal fluid, urine, etc. through the device. In certain embodiments, the device preserves the morphology and viability of non-disease-causing cells in the biological sample, e.g., red blood cells, lymphocytes or the like.

In any aspect or embodiment described herein, the device comprises an internal surface or internal volume that includes an adsorbent.

In certain embodiments, the internal surface is functionalized and linked or coupled to a ligand that selectively binds to the disease-causing agent, wherein the disease-causing agent is a cancer cell, e.g., a circulating cancer cell. In certain embodiments, the internal volume comprises an adsorbent that is functionalized and linked or coupled to a ligand that selectively binds to the disease-causing agent, wherein the disease-causing agent is a circulating cancer cell.

The functionalization of the adsorbent in the device can be performed by any method known in the art, including those described in co-pending US Patent Publication 2021-0106742, which is incorporated herein by reference in its entirety. In any aspect or embodiment described herein, the device for the at least one of the capture, isolation, identification, enumeration, measurement, detection, removal and/or destruction of the disease-causing agent comprises a hollow column or channel. In any aspect or embodiment described herein, the device comprises a hollow column or channel, and a reservoir.

In any aspect or embodiment described herein, the device comprises multiple hollow columns arranged in series. In any aspect or embodiment described herein, the device comprises multiple hollow columns arranged in parallel.

In any aspect or embodiment described herein, the device comprises a spiral or coiled hollow column or channel, and optionally a reservoir. In any aspect or embodiment described herein, the device comprises multiple hollow columns. In any aspect or embodiment described herein, the spiral column or channel has an inner diameter of from about 2 to about 10 mm (including, e.g., 3, 4, 5, 6, 7, 8, 9 mm and including all ranges therebetween). In any aspect or embodiment described herein, the spiral column or channel has a length of from about 2 to about 25 meters (including, e.g., 3, 4, 5, 6, 7, 8, 9, 10, 11, 12, 13, 14, 15, 16, 17, 18, 19, 20, 21, 22, 23, 24 m and including all ranges therebetween).

In any aspect or embodiment described herein, the spiral column or channel has volume of from about 5 to about 500 cc (including, e.g., 10, 20, 30, 40, 50, 60, 70, 80, 90, 100, 150, 200, 250, 300, 350, 400, and 450 cc and including all ranges therebetween).

In any aspect or embodiment described herein, the spiral column or channel has an inner surface area of from about 10 to about 200 cm² (including, e.g., 20, 30, 40, 50, 60, 70, 80, 90, 100, 110, 120, 130, 140, 150, 160, 170, 180, and 190 cm² and including all ranges therebetween).

In any aspect or embodiment described herein, the spiral column or channel has from 2 to 100 turns (including, e.g., 3, 4, 5, 6, 7, 8, 9, 10, 11, 12, 13, 14, 15, 16, 17, 18, 19, 20, 20, 25, 30, 35, 40, 45, 50, 55, 60, 65, 70, 75, 80, 85, 90, and 95 turns including all ranges therebetween) along its length from end to end.

In any aspect or embodiment described herein, the device has a length of from about 10 to about 500 mm (including, e.g., 10, 20, 30, 40, 50, 60, 70, 80, 90, 100, 150, 200, 250, 300, 350, 400, and 450 mm and including all ranges therebetween).

In any aspect or embodiment described herein, the device has a height of from about 2 to about 50 mm (including, e.g., 3, 4, 5, 6, 7, 8, 9, 10, 15, 20, 25, 30, 35, 40, 45, and 50 mm including all ranges therebetween).

In any aspect or embodiment described herein, the device has a distance between two consecutive spiral columns or channels of from about 2 to about 10 mm (including, e.g., 3, 4, 5, 6, 7, 8, and 9 mm and including all ranges therebetween).

In any aspect or embodiment described herein, the device has a total weight of beads within the column or channel of from about 10 to about 500 g (including, e.g., 15, 20, 25, 30, 35, 40, 45, 50, 55, 60, 65, 70, 75, 80, 85, 90, 95, 100, 150, 200, 250, 300, 350, 400, and 450 g and including all ranges therebetween).

It is expressly contemplated that any of the above elements can be combined with any of the other elements described herein in any combination.

In any aspect or embodiment described herein, the device is fabricated from at least one material selected from glass, steel, silicone, fluorinated polymers or a combination thereof.

In any aspect or embodiment described herein, the flow of the biological samples, e.g., fluid such as blood, serum, spinal fluid urine, etc. is in the laminar region.

In any aspect or embodiment described herein, the adsorbent is glass beads, glass microspheres or glass bubbles. In any aspect or embodiment described herein, the adsorbent is glass beads that are functionalized and linked to a ligand that selectively binds to the circulating cancer cells. In any aspect or embodiment described herein, the device comprises a ligand for the capture of CTCs from the biological sample, wherein the ligand binds specifically to a CTC biomarker selected from the group consisting of epithelial cell adhesion molecule (EpCAM), human epidermal growth factor receptor-2 (HER-2), epidermal growth factor receptor (EGFR), carcinoembryonic antigen (CEA), prostate specific antigen (PSA), CD24, and folate binding receptor (FAR).

In any aspect or embodiment described herein, the device comprises more than one ligand and/or more than one type of ligand that selectively binds to the cancer cell, e.g., circulating cancer cell (CTC). In any aspect or embodiment described herein, the sensitivity and specificity for the capture of CTCs is enhanced relative to the adsorbent alone. In any aspect or embodiment described herein, the device comprises an adsorbent configured for at least one of the capture, isolation, identification, enumeration, measurement, detection, removal, destruction or combination thereof, of MCF7 cells, A549 cells and CTCs.

In any aspect or embodiment described herein, the adsorbent glass beads are packed into the device, and the packed volume is in the range 10% to 90% v/v, including all values and ranges in between, e.g., 10%, 20%, 30%, 40%, 50%, 60%, 70%, 80%, 90% v/v.

In any aspect or embodiment described herein, the disease-causing agent is a circulating tumor cell, e.g., a cancer cell selected from a breast cancer cell, a prostate cancer cell, a colorectal cancer cell, a lung cancer cell, a pancreatic cancer cell, an ovarian cancer cell, a bladder cancer cell, an endometrine or uterine cancer cell, a cervical cancer cell, a liver cancer cell, a renal cancer cell, a thyroid cancer cell, a bone cancer cell, a lymphoma cell, a melanoma cell and a non-melanoma skin cancer cell, or a CTC derived from the above.

Methods

In additional aspects, the disclosure provides methods for at least one of the capture, isolation, identification, enumeration, measurement, detection, removal and/or destruction of a disease-causing agent from a biological sample of a cancer patient, e.g., a fluid such as blood, serum, spinal fluid, urine, etc. In any of the aspects or embodiments described herein, the method includes providing a device as described herein, and allowing the biological sample to flow through the device thereby capturing, isolating and/or removing the disease-causing agent, wherein the method is effective for the capture, isolation and/or removal of the disease-causing agent from the biological sample of the cancer patient. In any of the aspects or embodiments described herein, the method includes at least one additional step selected from identifying, enumerating, measuring, or detecting the disease-causing agent.

In any of the aspects or embodiments described herein, the biological sample, e.g., fluid such as blood, serum, spinal fluid, urine, etc. is passed through the device over a period of from about 5 minutes to about 60 minutes, including all values and ranges in between including, e.g., 5, 10, 15, 20, 25, 30, 35, 40, 45, 50, 55 or 60 minutes; and 5-55 minutes, 5-50 minutes, 5-45 minutes, 5-40 minutes, 5-35 minutes, 5-30 minutes, 5-25 minutes, 5-20 minutes, 5-15 minutes, 5-10 minutes; or 10-60 minutes, 15-60 minutes, 20-60 minutes, 25-60 minutes, 30-60 minutes, 35-60 minutes, 40-60 minutes, 45-60 minutes, 50-60 minutes.

In any of the aspects or embodiments described herein, the disease-causing agent is a circulating tumor cell, e.g., a cancer cell selected from a breast cancer cell, a prostate cancer cell, a colorectal cancer cell, a lung cancer cell, a pancreatic cancer cell, an ovarian cancer cell, a bladder cancer cell, an endometrine or uterine cancer cell, a cervical cancer cell, a liver cancer cell, a renal cancer cell, a thyroid cancer cell, a bone cancer cell, a lymphoma cell, a melanoma cell and a non-melanoma skin cancer cell or a CTC derived from the same.

In any of the aspects or embodiments as described herein, the method includes the step of treating the captured or isolated cancer cell with an anticancer drug, e.g., Vancomycin, Metformin, Doxorubicin, Methotrexate, Paclitaxel, 5-Fluorouracil, Cisplatin, and Camptothecin, Docetaxel, Oxaliplatin, and Cyclophosphamide, wherein the anticancer drug inhibits the growth, differentiation or induces apoptosis of the cancer cell.

In additional aspects, the disclosure provides methods for the capture, isolation, and destruction of a disease-causing agent from a biological sample of a cancer patient, e.g., a fluid such as blood, serum, spinal fluid, urine, etc. comprising the steps of providing a device as described herein, and allowing the biological sample to flow through the device thereby capturing or isolating the disease-causing agent, wherein the disease-causing agent is a cancer cell, and then treating the captured or isolated cancer cell with an anti-cancer drug. In any of the aspects or embodiments as described herein, anticancer drug is selected from, e.g., Vancomycin, Metformin, Doxorubicin, Methotrexate, Paclitaxel, 5-Fluorouracil, Cisplatin, and Camptothecin, Docetaxel, Oxaliplatin, and Cyclophosphamide, wherein the anticancer drug inhibits the growth, differentiation or induces apoptosis of the cancer cell.

In additional aspects, the disclosure provides methods of treating a cancer patient, comprising the steps of a) removing an amount (e.g., a fixed amount) of a biological sample, e.g., a fluid such as blood, serum, spinal fluid, urine, etc. from the cancer patient, b) passing the blood through a hemocompatible device as described herein for capturing or isolating a cancer cell, c) detecting the cancer cells present in the biological sample, captured or isolated after the passage of the biological sample through the hemocompatible device and measuring the number of cancer cells in the biological sample, and e) and infusing the treated blood back in to the cancer patient, wherein the method is effective for removing cancer cells from the blood of the patient but preserves the morphology and viability of the cancer patient's red blood cells.

In any aspect or embodiment described herein, the method includes prior to step (b), the step of estimating the number of cancer cells present in the amount of biological sample, e.g., a fluid such as blood, serum, spinal fluid, urine, etc. withdrawn by measuring the number of cancer cells in a test sample, e.g., in a sample of 1 to 1.5 milliliters.

In any aspect or embodiment described herein, the method includes prior to step (c), the step of estimating the number of the cancer cells present in the amount of biological sample, e.g., a fluid such as blood, serum, spinal fluid, urine, etc. recovered after the passage of the biological sample through the hemocompatible device by measuring the number of cancer cells in a sample of 1 to 1.5 milliliters.

In any aspect or embodiment described herein, the biological sample is blood. In any of the aspects or embodiments, the disease-causing agent or cancer cell is a circulating tumor cell.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the present disclosure may be utilized in numerous combinations, all of which are expressly contemplated by the present description. These additional advantages, objects and embodiments are expressly included within the scope of the present disclosure. The publications and other materials used herein to illuminate the background of the disclosure, and in particular cases, to provide additional details respecting the practice, are incorporated by reference in their entirety for all purposes.

As used herein, the term ‘functionalizing agent’ refers to a molecule that attaches itself to a substrate through a functional group non-covalently to modify the characteristics of the substrate.

As used herein, the term ‘ligand’ refers to a molecule that is covalently linked to the functionalizing agent at one end and non-covalently attaches itself to the moiety in the blood, which is to be at least one of captured, isolated, detected, measured, enumerated, accounted, removed or destroyed.

As would be understood by the skilled artisan in view of the present disclosure, numerous modifications can be made to designs of devices as described herein, and methods of using the same, which would achieve adsorption of cancer causative or non-hematopoietic substances from the blood (e.g. CfDNA, ctDNA, exosomes, drugs, miRNA), which are intended and contemplated by the present description.

The invention is now illustrated by way of non-limiting examples, which are to be regarded as illustrative in nature and do not limit the scope of invention in any manner.

### EXAMPLES

Computational fluid dynamics (CFD) has been used extensively as a tool to design hemodynamic devices. CPD analysis was used to select the device and process parameters on for efficient capture of disease-causing cells. The effect of device and process variables on the performance of the device in terms of: 1) pressure drop, 2) hemolysis index, 3) thrombosis, 4) number of CTCs captured, 5) cost efficiency of capture, 6) geometric probability of capture was determined.

The CFD analysis was carried out using CFX v19 (ANSYS Inc, Pittsburgh, PA). A tetrahedral mesh with a prism boundary layer of first layer thickness 8 μm was used. The mesh consisted of total of 86610 nodes and 319532 elements and mesh independence was studied and satisfied. Inlet boundary condition of flow rate of, e.g., about 100 milliliters/min, was used and a static pressure condition of 70 mmHg was considered at the outlet of the channel. In certain embodiments, the flow rate ranges from about 1 to about 500 milliliters/min, e.g., about 1, about 5, about 10, about 15, about 20, about 25, about 30, about 35, about 40, about 45, about 50, about 55, about 60, about 65, about 70, about 75, about 80, about 85, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, or about 500 milliliters/min.

A laminar model was used, and the working fluid was assumed to be incompressible and Newtonian. The solution was assumed to be converged when the residuals achieved were less than 10⁻⁵. A high-resolution scheme was used.

The CFD analysis was carried out as to yield a hemolysis index in the range of 10⁻⁵ to 10⁻²% for population of 25 beads spaced at 0.5 mm which yielded bead diameter in the range 0.5-3.5 mm.

The maximum flow rate during hemodialysis was maintained at 500 milliliters/min. This flow rate was defined on the basis of the maximum flow rate for the device and was expected to operate at physiological flow of blood which is mostly in the laminar region, having Reynolds number of 850. The tube diameter corresponding to this Reynold number was of the order 4 mm. The analysis was also used to evaluate the number of CTCs captured.

Hemolysis assay using device was carried out as described below:

Red Blood Cells from blood of the cancer patient were separated by centrifugation at room temperature and washed in sodium chloride solution two times and resuspended in phosphate buffer saline, pH 7.4. The resuspended red blood cells were incubated with the composition to be evaluated for 30 minutes at 37° C. The samples were centrifuged, and the supernatant collected and analyzed for hemolysis using UV spectroscopy. The percentage hemolysis was estimated against a negative control phosphate buffer saline, pH 7.4 and positive control 0.5% Triton-X100 prepared in phosphate-buffered saline, pH 7.4.

Human colon cancer cells (HCT116) and MCF7 cell capture using the device was carried out as described below:

MCF 7 and A549 cells were incubated in a channel device containing glass beads of Example 12 for 5 minutes. The cells were isolated, enriched, fixed and stained with antibodies against cytokeratin (CK18) and leucocyte common antigen (CD 45) and counterstained with 4′,6-diamidino-2-phenylindole (DAPI). Isolated cells were imaged using Zeiss fluorescence scope.

The capture of Circulating Tumor Cells (CTC) from cancer patient's blood using device was carried out as described below:

The cancer patient's blood was incubated in spiral channel device containing glass beads of Example 12. CTCs bound to these glass beads were isolated. The captured circulating tumor cells were fixed and stained with antibodies against cytokeratin (CK-18) and leucocyte common antigen (CD 45) and counterstained with 4′,6-diamidino-2-phenylindole (DAPI). Isolated cells were imaged using Zeiss fluorescence scope and were characterized as cells that are designated as CK-18⁺, DAPI⁺ and CD45⁻.

### Example 1

Spiral Channel Device

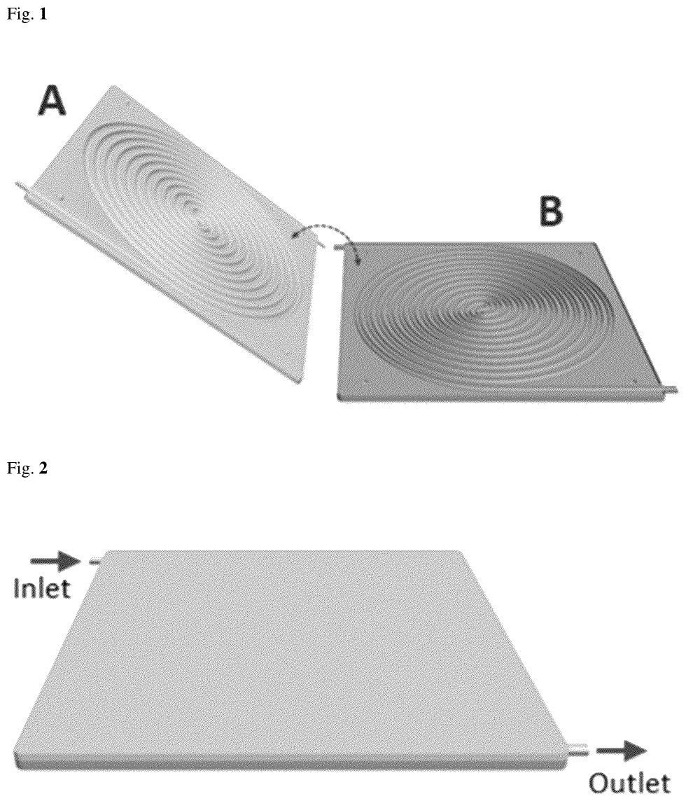

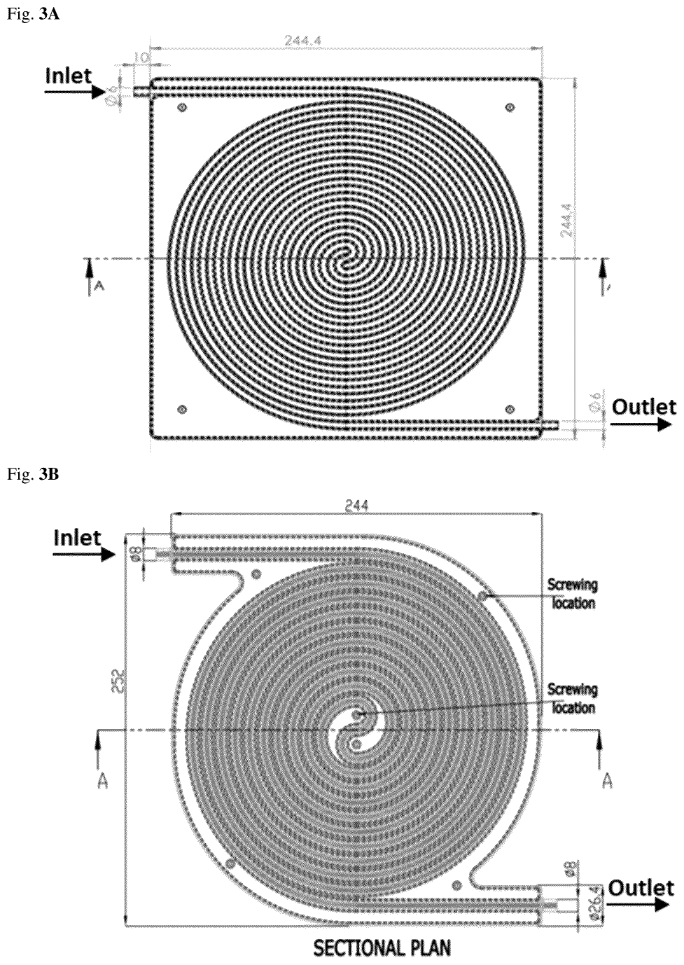

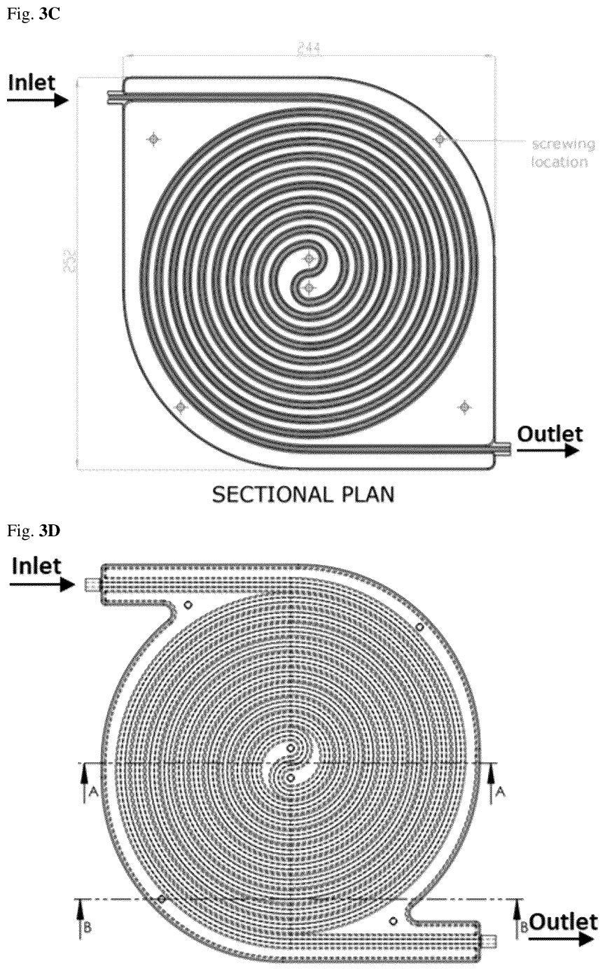

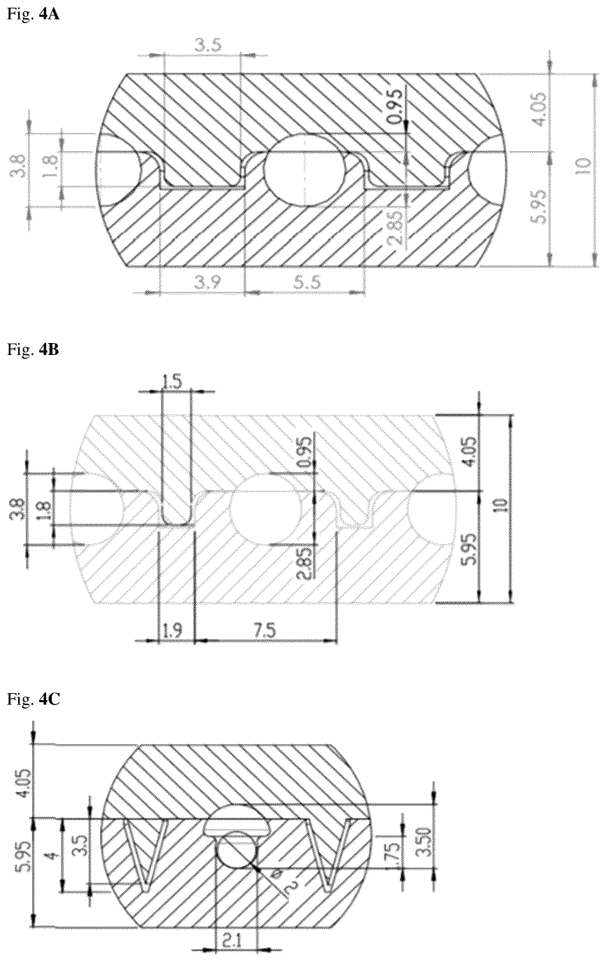

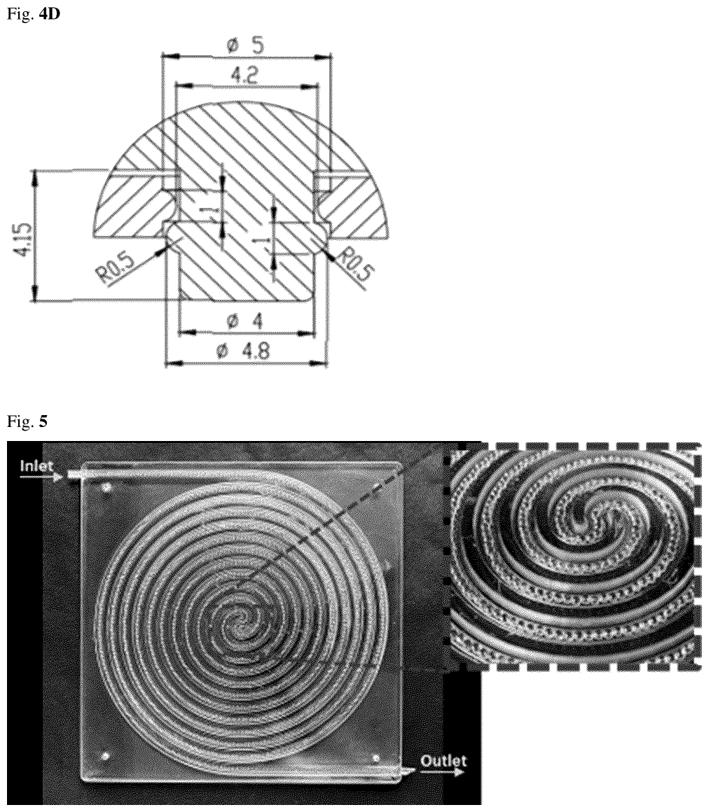

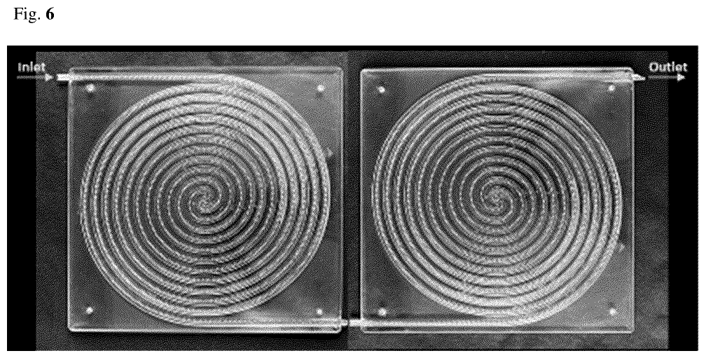

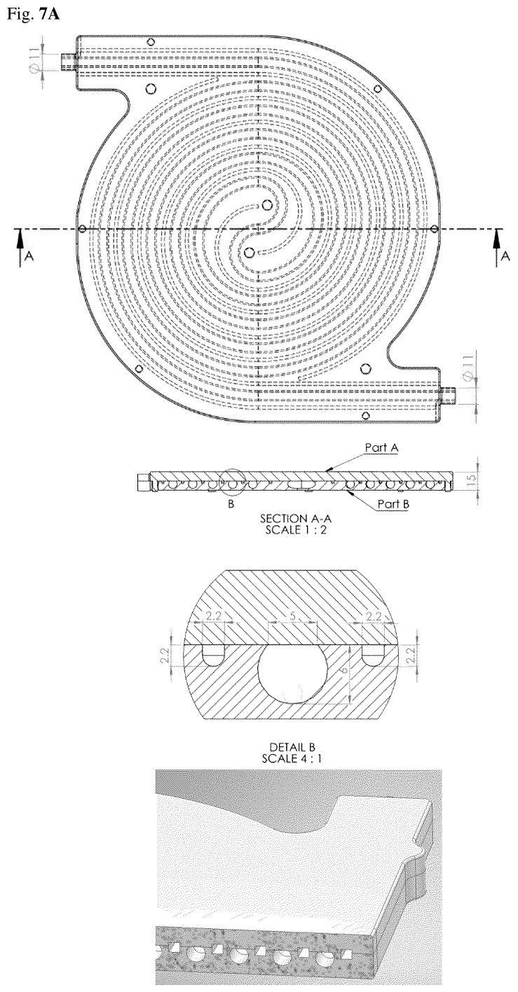

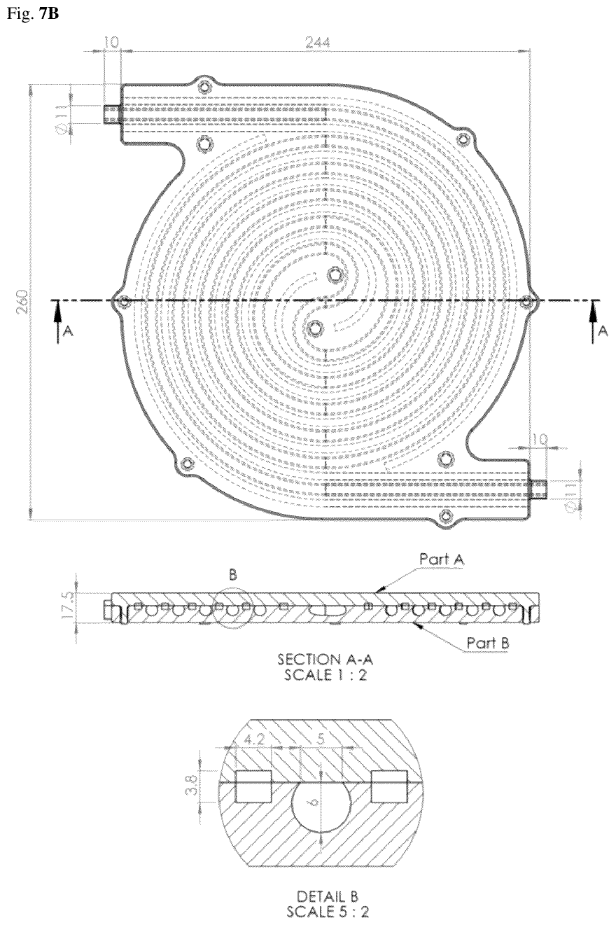

The device was fabricated using different medical grade materials, such as silicone, steel, glass, fluorinated polymers. The device consists of two parts—A and B, overlayed together (FIG. 1, 2, 3A, 3B, 3C, 3D, 4A, 4B, 4C, 4D, 5, 6 and -7). To those skilled in the art, it would be apparent that the specifications of spiral channel device similarly can be designed having inner diameter of channel 2-10 mm, length 2-25 meters, volume 5-500 cc, inner surface area of channel 10-200 square cm, and number of turns of spiral channel 2-100 turns. Device specifications are mentioned in Table 1.

TABLE 1
Specifications of spiral channel device 1.

| Sr. | | | |
|---|---|---|---|
| No. | Specifications | Measure | |
| 1. | Inner diameter of channel | 3.8 | mm |
| 2. | Length | 6.8 | meters |
| 3. | Volume | 77 | cc |
| 4. | Inner surface area of channel | 81 | square cm |
| 5. | Number of turns of spiral channel | 21 | turns |
| 6. | Length device | 244.4 | mm |
| 7. | Height of device | 10 | mm |
| 8. | Distance between two consecutives spiral | 3.9 | mm |
| | channels | | |
| 9. | Weight of whole device (A-Lid and B-base | 630 | g |
| | plate) | | |
| 10. | Weight of top lid A plate | 338 | g |
| 11. | Weight of base B plate | 292 | g |
| 12. | Total weight of the beads required to fill the | 28 | g |
| | device | | |

### Example 2

Computational Fluid Dynamic Analysis of Spiral Channel Device Filled with Surface Functionalized Glass Beads.

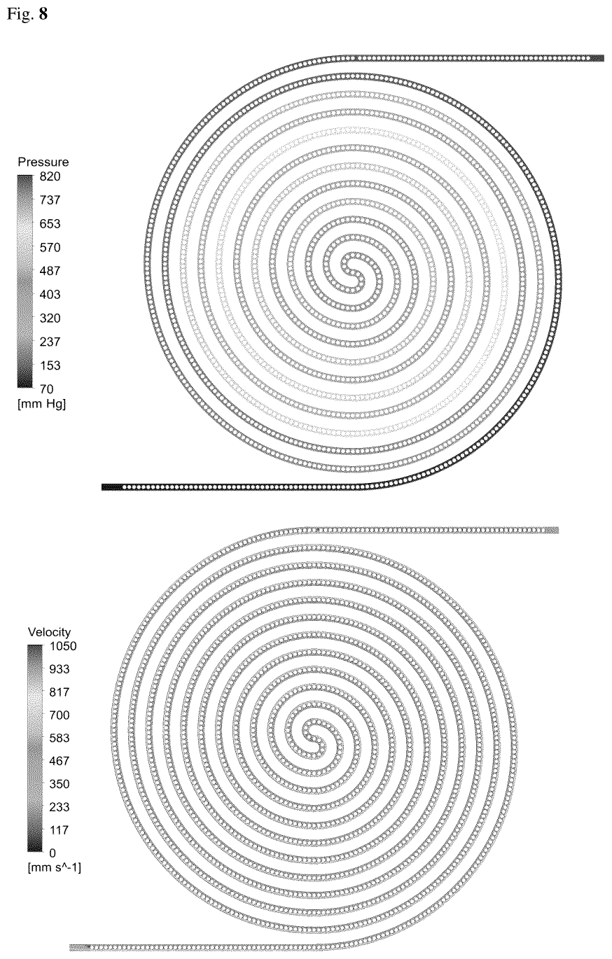

3D model of spiral channel device filled with surface functionalized glass beads was analyzed using Ansys software (FIG. 8). Fluid velocity and pressure were analyzed throughout.

Keeping all other conditions and geometrical parameters constant, the bead diameter was varied. The hemolysis index of VADs varied from ≈10⁻³ to 10⁻²%. In the current simulation, only 25 beads were considered. With increase in the number of beads, the hemolysis index increased proportionately.

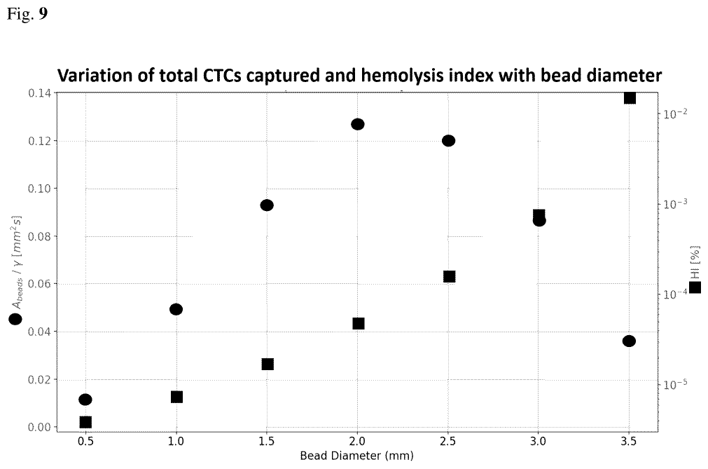

As seen in FIG. 9 the HI increases exponentially with increase in bead diameter (the HI axis is a log scale). To target ≈2500 beads to be used for the device and keep the HI below 10⁻², the HI for 25 beads will have to be less than 10⁻⁴. Thus, beads of 2 mm diameter provide a HI % of less than 10⁻⁴.

Additionally, as seen in FIG. 9, A_{beads}/γ reaches a peak at 2 mm and then start declining with increase in bead diameter. Thus, the total CTCs capture will reach a maxima at a bead diameter of 2 mm.

### Example 3

Estimation of Blood Flow Velocity Through Empty Spiral Channel Device.

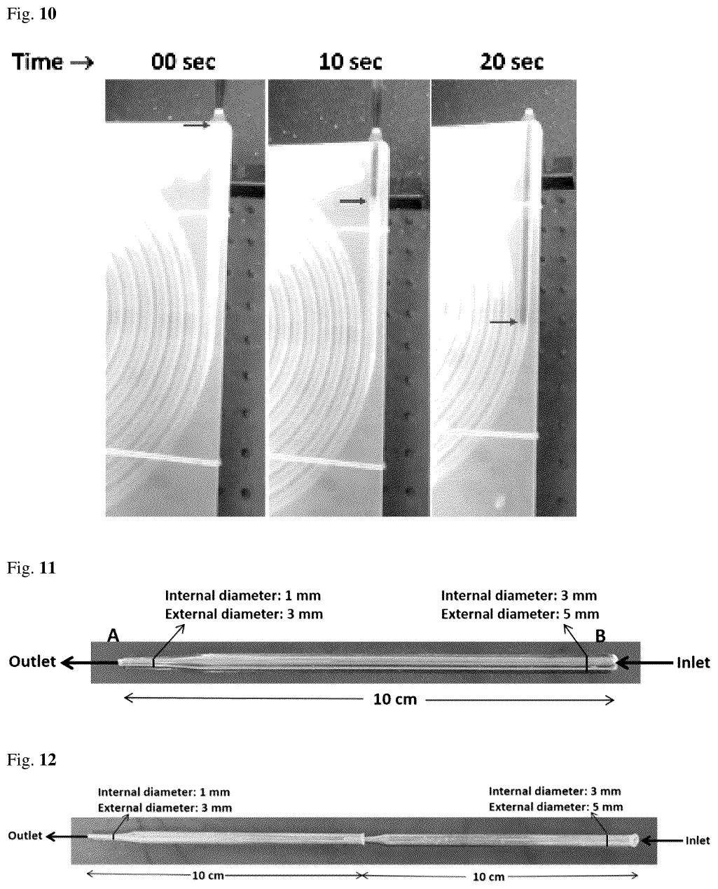

1 milliliter of cancer patient's whole blood was pipetted into the inlet channel of the spiral channel device and was allowed to flow through the channel under capillary action. A video of the blood flow through the channel was recorded. The velocity of the blood through the channel was calculated using Image J software. Blood flow velocity was 5.7 mm/second in the inlet channel of spiral channel device (FIG. 10).

### Example 4

A Hollow Cylindrical Column.

The device was fabricated using a borosilicate glass tube having a length of 10 cm from end A to end B with an internal diameter of 3 mm and an external diameter of 5 mm (FIG. 11). At end A, the column tapers with an internal diameter of 1 mm and an external diameter of 3 mm. These cylindrical glass columns in series were arranged horizontally (FIG. 12) or arranged in parallel connected using medical grade silicone tubing (FIG. 13).

The retention volumes of different fluids viz. PBS and blood in device were measured. The volume of PBS occupied, volume of blood occupied, volume of PBS retained, and volume of blood retained were 1 milliliter, 0.9 milliliter, 0.05 milliliter and 0.05 milliliter respectively.

### Example 5

A Hollow Cylindrical Column Containing a Reservoir.

The device was fabricated using a borosilicate glass tube of length 15 cm with an internal diameter of 3 mm and an external diameter of 5 mm, having a reservoir of length 5 cm and an internal diameter of 2 cm (FIG. 14). At one end, the column tapers with an internal diameter 1 mm and an external diameter 3 mm.

The retention volumes of different fluids viz. PBS and blood in device (FIG. 14) were measured. The volume of PBS occupied, volume of blood occupied, volume of PBS retained and volume of blood retained were 13.8 milliliters, 11.8 milliliters, 0.1 milliliter and 0.1 milliliter respectively.

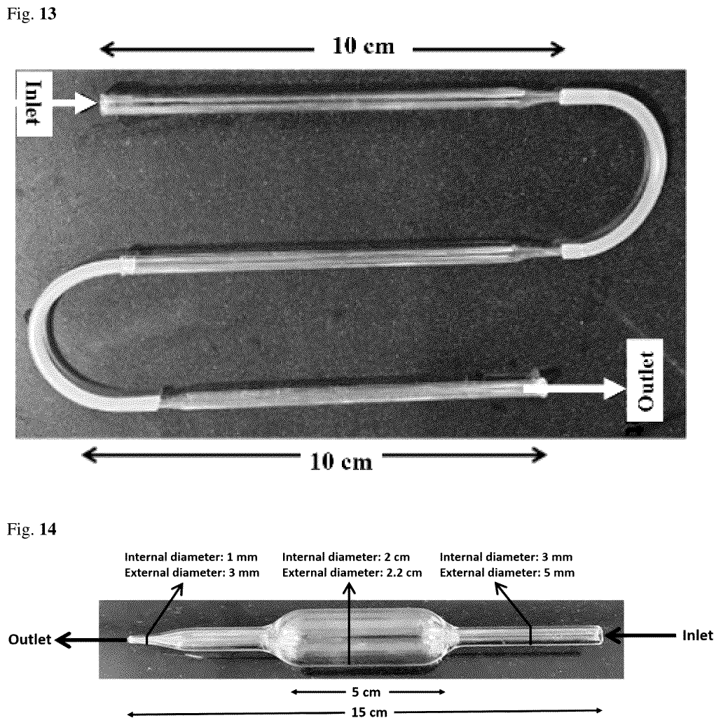

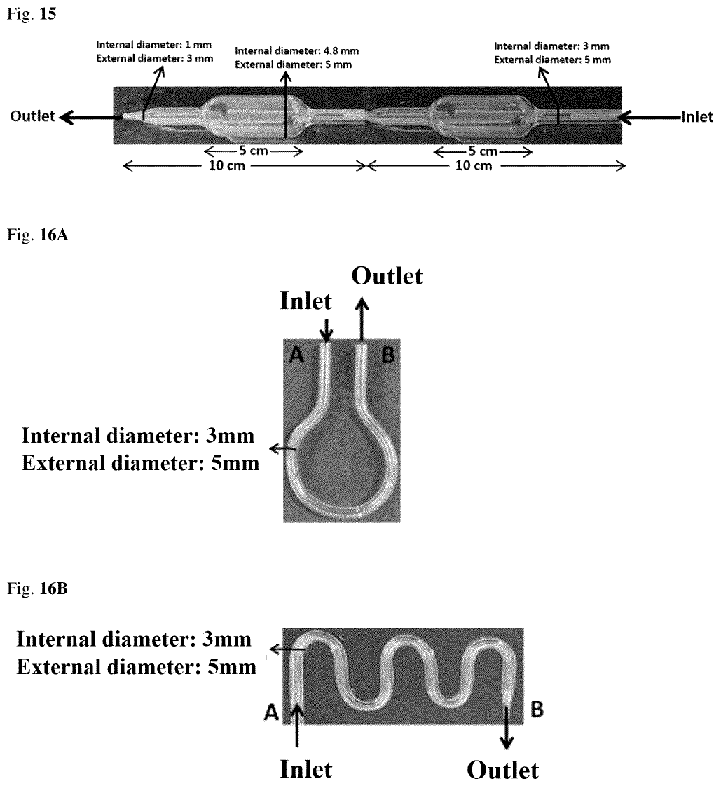

The length of the column was varied from 5 cm to 25 cm. The length of the reservoir was varied from 5 cm to 10 cm. The columns were used as a single unit or in series (FIG. 15). The columns were arranged horizontally, vertically or at different angles. The columns were either stationary or in motion.

The column material was selected from glass, steel, silicone and fluorinated polymers.

### Example 6

A Hollow U-Shaped Column.

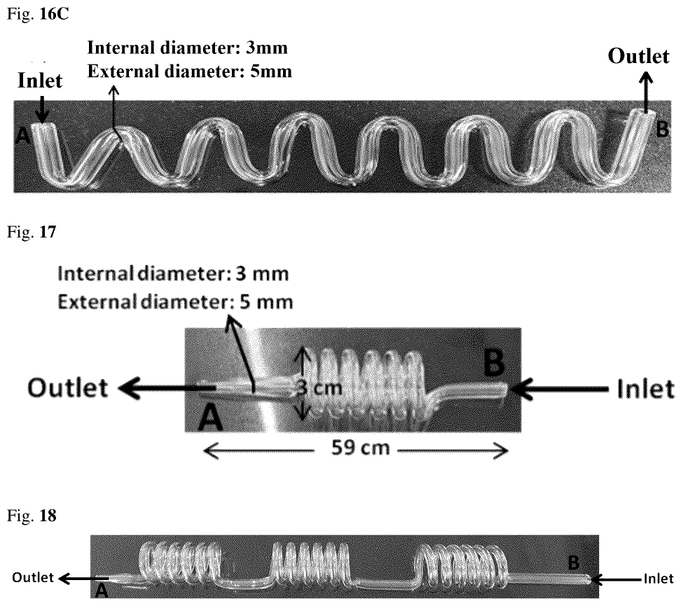

The device was fabricated from a borosilicate glass tube having a length 37 cm from end A to end B, with internal diameter of 3 mm and an external diameter of 5 mm (FIGS. 16A, 16B, and 16C). The length of the tube chosen was varied from 34 cm to 100 cm. The columns were used as a single unit or in series (FIGS. 16B and 16C). The columns were arranged horizontally or vertically or at different angles. The columns were either stationary or in motion. The material of the column was selected from glass, silicone, steel, fluorinated polymers.

The retention volumes of different fluids viz. PBS and blood in device (FIG. 16A) were measured. The volume of PBS occupied, volume of blood occupied, volume of PBS retained and volume of blood retained were 2 milliliters, 1.8 milliliters, 0.05 milliliter and 0.05 milliliter respectively.

### Example 7

A Hollow Coiled Column

The device was fabricated using a borosilicate glass tube and had a length 59 cm from end A to end B, the internal diameter of glass tube was 3 mm, and the external diameter was 5 mm (FIG. 17). The internal diameter of the coiled column was 2 cm. The length of the coiled glass column was varied from 59 cm to 120 cm. The internal diameter of the column was varied from 2 cm to 12 cm. The columns were used as a single unit or in series. The columns were arranged horizontally or vertically or at different angles. The columns were either stationary or in motion. The material of the column was selected from glass, silicone, steel, and fluorinated polymers.

The retention volumes of different fluids viz. PBS and blood in device (FIG. 17) were measured. The volume of PBS occupied, volume of blood occupied, volume of PBS retained and volume of blood retained were 6 milliliters, 1.2 milliliters, 0.1 milliliter and 0.1 milliliter respectively.

A hollow coiled column can also contain more than one coiled elements in series as shown in FIG. 18.

### Example 8

A Hollow Spiral Column.

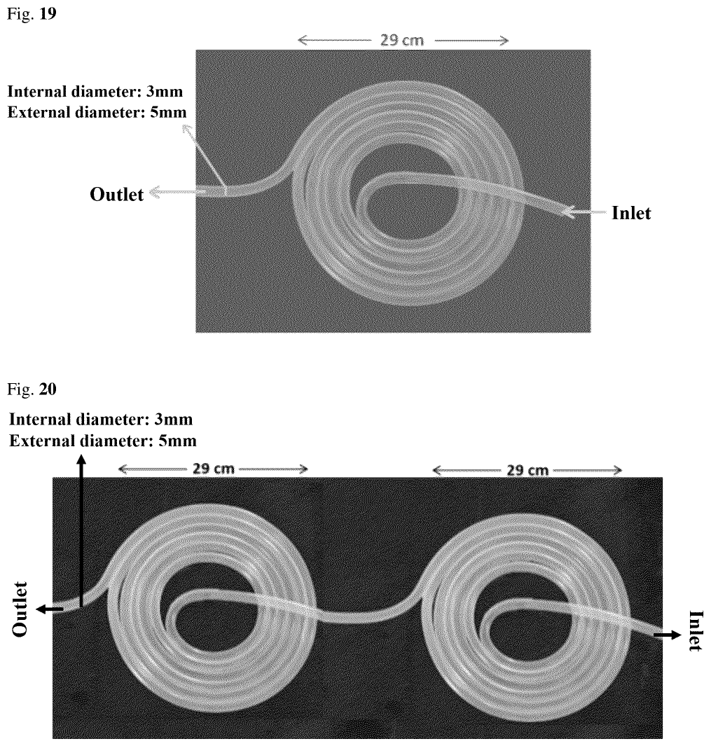

The device was fabricated using a silicone tube of length 71 cm, with an internal diameter of 3 mm and an external diameter of 5 mm (FIG. 19). The outer diameter of the silicone column was 29 cm.

The length of the silicone tube was varied from 17 cm to 120 cm and the outer diameter of the column was varied from 5 cm to 35 cm. The columns were used as a single unit or in series. (FIGS. 19 and 20). The columns were arranged horizontally or vertically or at different angles. The columns were either stationary or in motion. The material of the column was selected from glass, silicone, steel and fluorinated polymers.

The retention volumes of different fluids viz. PBS and blood in device were measured. The volume of PBS occupied, volume of blood occupied, volume of PBS retained, and volume of blood retained were 7.75 milliliters, 7.0 milliliters, 0.1 milliliter and 0.1 milliliter respectively.

### Example 9

Cylindrical channel device design for continuous blood flow with 4 parallel spiral paths.

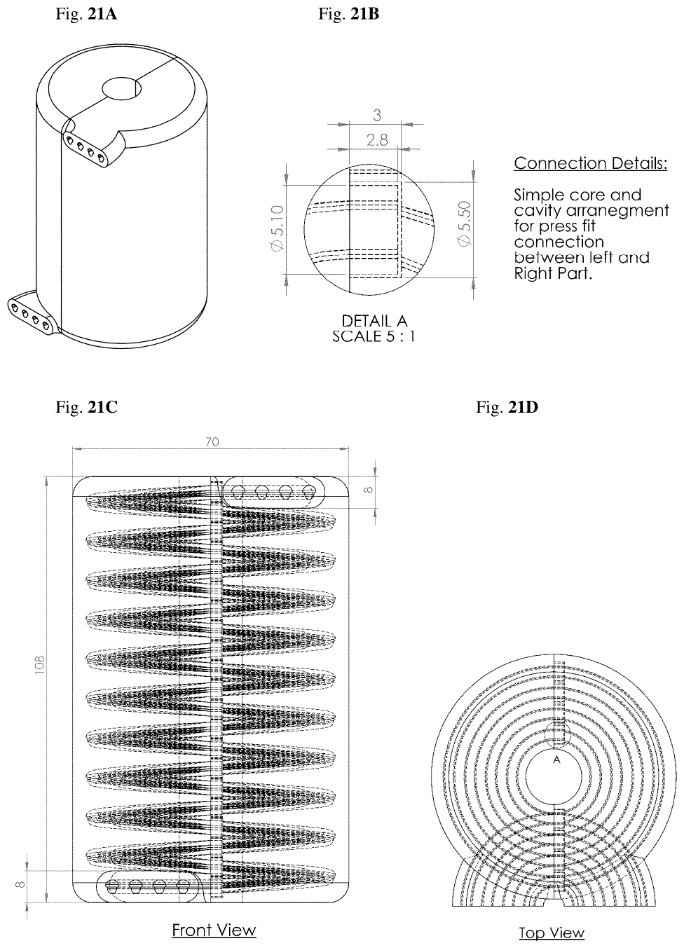

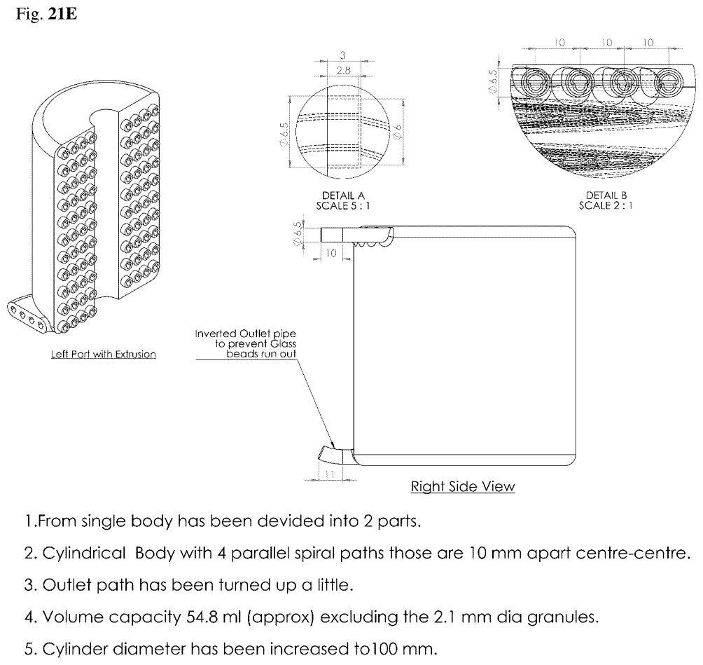

The device was fabricated using different medical grade materials such as silicone, fluorinated polymers, and steel. Device having different dimensions and constructions and two separate detachable sections are represented in FIGS. 21A-21E.

### Example 10

Functionalization of Glass Column with (3-glycidyloxypropyl)trimethoxysilane.

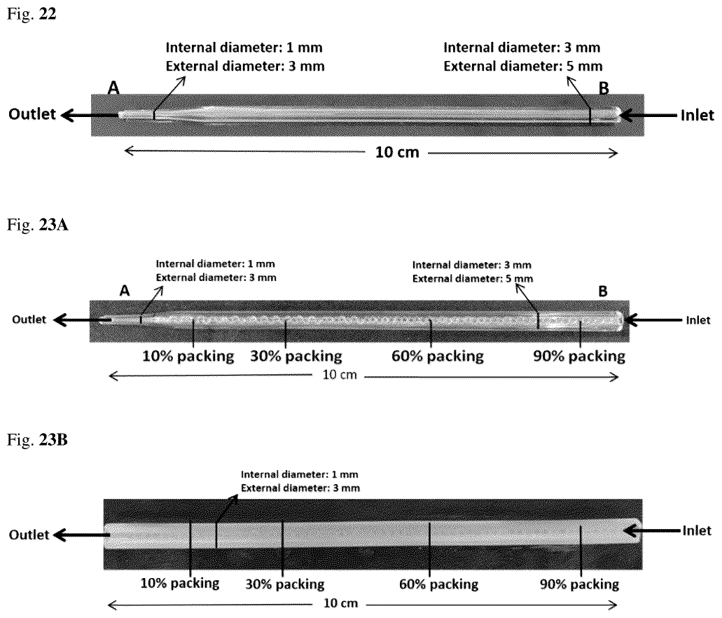

A hollow glass column selected from example 4-7 was filled with 1 milliliter distilled water containing 0.16 gram of sodium hydroxide and treated for 10 minutes. The water was drained off and the column was washed with 1 milliliter distilled water three times and dried in an oven at 100° C. for an hour. The inner surface of the column was treated with piranha solution for 3 hours. The piranha solution was then drained, and the column was washed with 1 milliliter water each six times. The column was dried in a hot air oven at 100° C. for 2 hours. The dried column was then treated with a solution of 0.1 milliliter (3-glycidyloxypropyl)trimethoxysilane in 0.9 milliliter toluene for 30 minutes at room temperature. The unreacted (3-glycidyloxypropyl) trimethoxysilane in toluene was drained off and the column was washed with 1 milliliter toluene followed by 1 milliliter acetone and finally with 1 milliliter ethanol. The salinized hollow cylindrical glass column was dried in a hot air oven at 110° C. for 6 hours (FIG. 22).

To those skilled in the art, it would be apparent that the reaction can be carried out suitably using different devices (FIGS. 11-18).

### Example 11

Linking of Anti-Epithelial Cell Adhesion Molecule with (3 glycidyloxypropyl) trimethoxysilane Functionalized Glass Column.

The (3-glycidyloxypropyl)-trimethoxysilane functionalized hollow cylindrical glass column of example 10 was treated with a solution of 2 μg of anti-epithelial cell adhesion molecule (EpCAM) antibody in 1 milliliter phosphate-buffered saline pH 7.9 for 4 hours at 4° C. The phosphate-buffered saline was drained, and the glass column was washed with phosphate-buffered saline pH 7.4 three times and filled with phosphate-buffered saline pH 7.4 and stored at 4° C. (FIG. 22). To those skilled in the art, it would be apparent that the reaction can be carried out similarly using different devices (FIG. 11-19).

### Example 12

Linking of (3-glycidyloxypropyl)Silane Functionalized Glass Beads with Anti-Epithelial Cell Adhesion Molecule Antibody.

100 milligrams glass beads were functionalized as described in example 10, were treated with 1 microgram of anti-epithelial cell adhesion molecule antibody in 200 microliter of phosphate buffered saline pH 7.2 for 4 hours at 4° C. The glass beads were washed with 1 milliliter phosphate buffered saline pH 7.2 thrice and finally stored in 0.2 milliliter phosphate buffered saline pH 7.2 at 4° C.

The linking of anti-epithelial cell adhesion molecule antibody to (3-glycidyloxypropyl)silane functionalized glass bead was confirmed by fluorescein isothiocyanate. The fluorescein isothiocyanate intensity of anti-epithelial cell adhesion molecule antibody linked to glass beads was 987.37 which was four times higher than that for (3-glycidyloxypropyl)silane functionalized glass bead sans anti-epithelial cell adhesion molecule antibody (241.64). The increase in intensity confirmed the linking of anti-epithelial cell adhesion molecule antibody to (3-glycidyloxypropyl)silane functionalized glass bead.

The linkage of (3-glycidyloxypropyl)silane functionalized glass beads to anti-epithelial cell adhesion molecule antibody was confirmed by the CTC and HCT-116 capturing assay.

It would be apparent to the persons skilled in the art that ligands which can bind to the target transferrin, bovine serum albumin, N-acetyl glucosamine and other active biomolecules can also be linked to glass beads by varying the conditions of linking.

### Example 13

A Hollow Cylindrical Glass Column Packed with Glass Beads of Example 12.

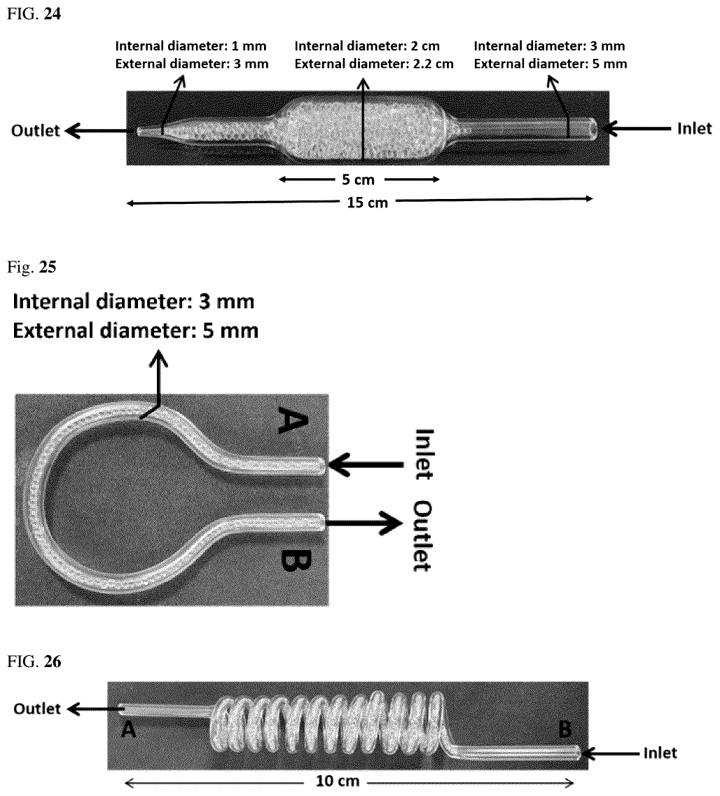

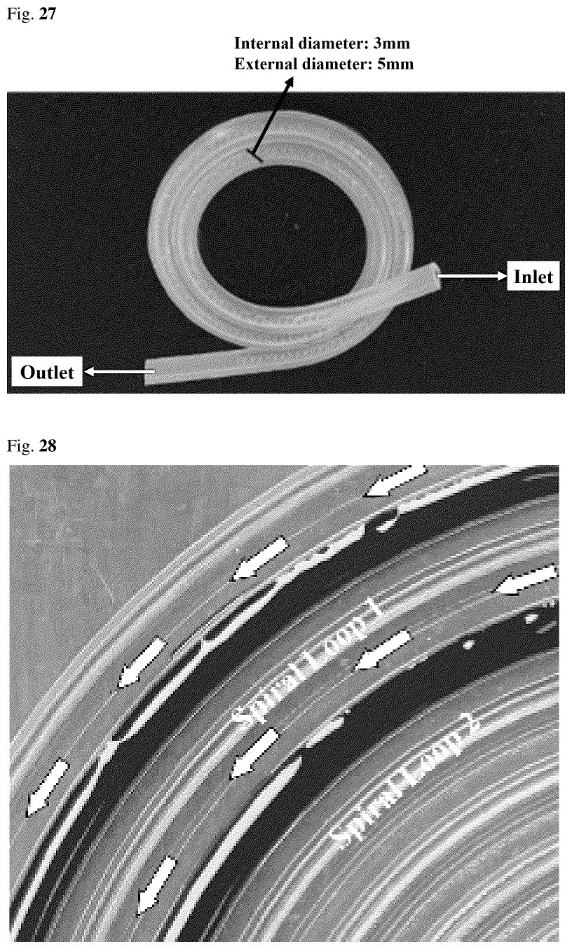

Hollow glass columns of example 4-8 were packed with glass beads of example 12. The packing volume of the hollow cylindrical column was varied between 10-90% (FIGS. 23A, 23B, and 24-27). The material of the column could also be selected from glass, silicone, and fluorinated polymers.

### Example 14

Retention of Fluids and Blood in Spiral Channel Device.

The retention volumes for the device of example 1 were measured using fluids such as phosphate-buffered saline and blood. The device was completely filled with a known volume of fluids and cancer patient's blood using a syringe pump. The fluid was then allowed to be emptied under gravitational pull. The difference between the total volumes filled and the volume of fluid that was vacated under the gravitational pull was the retention volume. Retention volumes for PBS, as well as blood, was 50 μl for hollow cylindrical glass column The retention volume was maximum for spiral glass/silicone column, 0.1 milliliter each. The retention volumes for each column configuration for the device have been listed in Table 2.

TABLE 2
Results of retention volume for different fluids.

| | | | Volume | Volume | Volume | Volume |
|---|---|---|---|---|---|---|
| Column | Internal | | PBS | blood | PBS | blood |
| configuration | diameter | Length | occupied | occupied | retained | retained |
| FIG. No. | (mm) | (cm) | milliliter | milliliter | milliliter | milliliter |
| FIG. 11 | 3 | 10 | 1 | 0.9 | 0.05 | 0.05 |
| FIG. 14 | 5 (stem), | 10 | 13.8 | 11.8 | 0.1 | 0.1 |
| | 2 cm | | | | | |
| | (reservoir) | | | | | |
| FIG. 16C | 3 | 37 | 2 | 1.8 | 0.05 | 0.05 |
| FIG. 17 | 3 | 59 | 6 | 1.2 | 0.1 | 0.1 |
| FIG. 19 | 3 | 29 | 7.75 | 7.00 | 0.1 | 0.1 |

All columns were made from glass, except that in the last row which was made of silicone.

### Example 15

Red Blood Cell Hemolysis Assay.

Red blood cells from healthy person's blood (5 milliliters) were separated by centrifugation (500×g, 5 minutes) at room temperature and washed with 150 mM sodium chloride (NaCl) solution two times, and resuspended in 5 milliliters phosphate-buffered saline, pH 7.4. The resuspended red blood cells were incubated on the spiral channel device 1 described in Example 1 for 5 to 60 minutes at room temperature and with mild agitation on a rocker shaker. The samples were centrifuged (500×g, 5 minutes, room temperature) and supernatants were collected and analyzed for hemolysis by UV spectroscopy at 540 nm. The percentage hemolysis was estimated against a negative control phosphate-buffered saline, pH 7.4 and positive control 0.5% Triton-X 100. The percent hemolysis is shown in Table 3.

TABLE 3
Percentage hemolysis with Red Blood Cells on spiral
channel device as described in example 1.

| Sr. No. | Sample and Incubation Time (in minutes) | Hemolysis |
|---|---|---|
| 1. | Phosphate Buffered Saline | 0% |
| 2. | Triton-X 100 (0.5%) | 100% |
| 3. | 5 Minute on-device incubation | 0% |
| 4. | 15 Minute on-device incubation | 0% |
| 5. | 30 Minute on-device incubation | 0% |
| 6. | 45 Minute on-device incubation | 0% |
| 7. | 60 Minute on-device incubation | 0% |

### Example 16

Red Blood Cell Hemolysis Assay and Morphology of Cells Treated with Glass Beads.

Red blood cells from cancer patient's blood (5 milliliters) were separated by centrifugation (500×, 5 minutes) at room temperature and washed in 150 mM sodium chloride (NaCl) solution two times, and resuspended in 5 milliliters, phosphate-buffered saline pH 7.4. The resuspended red blood cells were incubated (FIG. 28) by mixing with materials listed in Table 3 for 30 minutes at 37° C. The samples were centrifuged (500×g, 5 minutes, room temperature) and supernatants were collected and analyzed for hemolysis using UV spectroscopy at 540 nm. The percent hemolysis is summarized in Table 4.

TABLE 4
Percentage hemolysis of red blood cells using raw materials,
substrates and intermediates utilized in compositions.

| Sr. | Sample | Hemolysis |
|---|---|---|
| 1. | Phosphate Buffered Saline | 0% |
| 2. | Triton-X 100 (0.5%) | 100% |
| 3. | Unwashed Glass Beads (2 mm) (~200 milligrams) | 0.9% |
| 4. | Washed Glass Beads (2 mm) (GB) (~200 milligrams) | 0% |
| 5. | 20% (3-Glycidyloxypropyl)trimethoxysilane (GPTMS) | 100% |
| 6. | Glass Beads treated with 50% GPTMS | 0% |
| 7 | Glass beads of example 12 | 0% |

FIG. 29 shows the RBC morphologies for blood treated with glass beads and controls.

### Example 17

Capture of Breast Cancer (MCF7) Cells Using Spiral Channel Device.

Spiral channel device of example 1 was packed with glass beads of example 12.

MCF7 cell suspension in 500 μl of Complete Minimum Essential Medium Eagle media containing 500 cells/milliliter was passed through spiral channel device and beads inside the device were analyzed for cell capture. Cells captured were fixed with 1 milliliter of absolute methanol and immune-stained with anti-cytokeratin (CK-18) antibodies and with nuclear-staining probe 4′, 6-diamidino-2-phenylindole (DAPI).

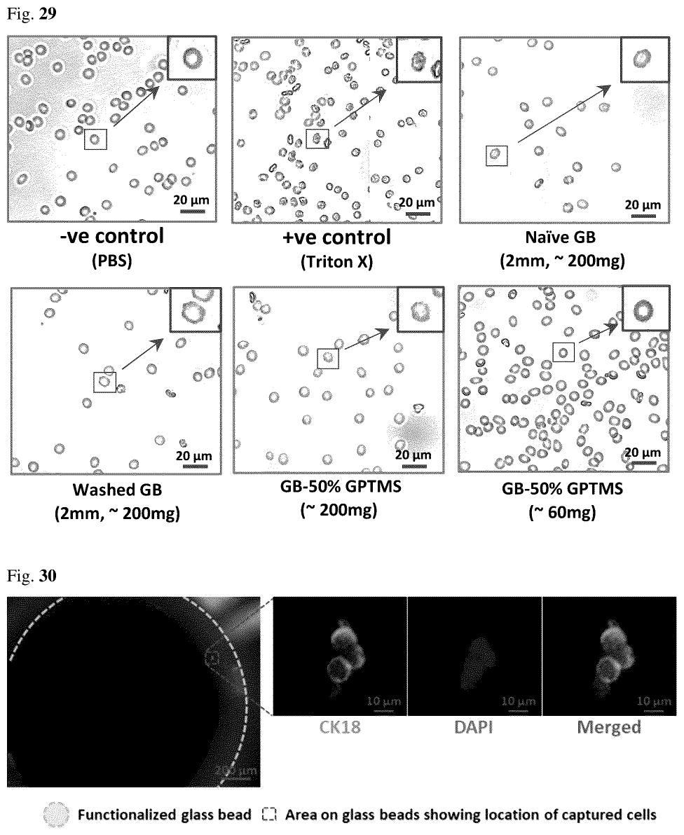

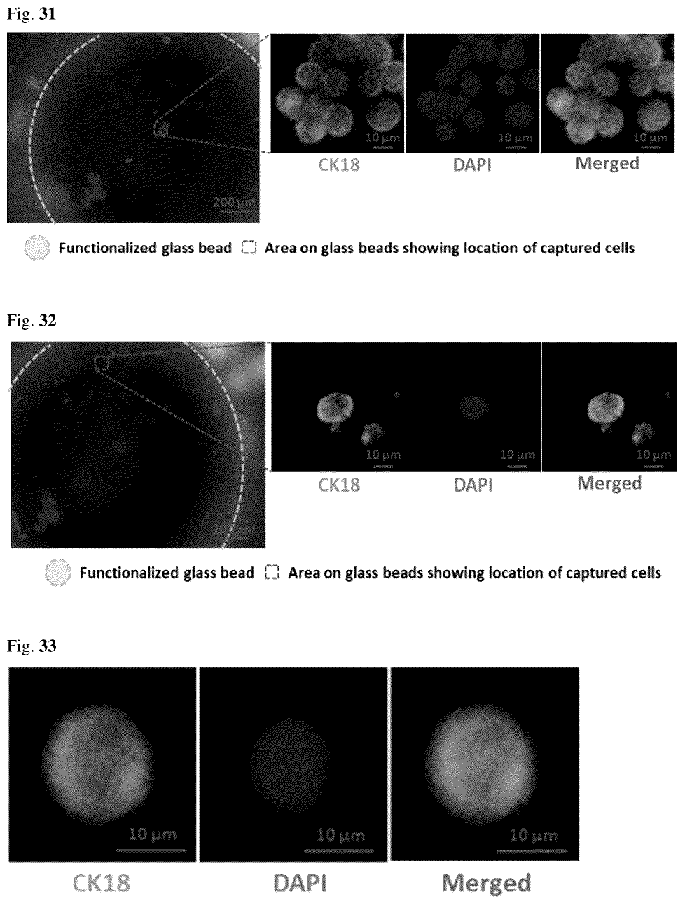

Captured cells and clusters were observed and imaged under fluorescence scope (FIGS. 30 and 31).

### Example 18

Capture of Lung Cancer (A549) Cells Using Spiral Channel Device.

Spiral channel device of example 1 was packed with glass beads of example 12. Cell suspension in 500 μl of Complete Ham's F-12K (Kaighn's) medium containing 500 cells/milliliter was passed through spiral channel device and beads inside the device were analyzed for cell capture.

Cells captured were fixed with 1 milliliter of absolute methanol and immune-stained with anti-cytokeratin (CK-18) antibodies and with nuclear-staining probe 4′, 6-diamidino-2-phenylindole (DAPI).

Captured cells were observed and imaged under fluorescence scope (FIG. 32).

### Example 19

Capture of Breast Cancer (MCF7) Cells and Lung Cancer (A549) Cells Using Series of Spiral Channel Devices.

MCF7 cell suspension in 500 μl of Complete Minimum Essential Medium Eagle media containing 500 cells/milliliter was passed through series of 3 spiral channel device of Example 1 containing three glass beads of example 12 in each device. The beads from each device were then treated with trypsin to detach the captured cells. Cells detached from beads were fixed with 1 milliliter of absolute methanol and immune-stained with anti-cytokeratin (CK-18) antibodies and with nuclear-staining probe 4′, 6-diamidino-2-phenylindole (DAPI). Captured cells were observed and imaged under fluorescence scope (FIGS. 33 and 34).

Cells captured on each bead and the capture percentage in each device were also accounted and the results are summarized in Table 5.

TABLE 5
Percent cells captured on each bead in each device.

% Cells captured

| | | | |
|---|---|---|---|
| No. | Bead numbers | MCF7 cells | A549 cells |

First

| | | | |
|---|---|---|---|
| 1. | First | 44 | 23 |
| 2. | Second | 11 | 28 |
| 3. | Third | 09 | 19 |

Second

| | | | |
|---|---|---|---|
| 1. | First | 08 | 04 |
| 2. | Second | 12 | 06 |
| 3. | Third | 04 | 05 |

Third

| | | | |
|---|---|---|---|
| 1. | First | 02 | 00 |
| 2. | Second | 00 | 02 |
| 3. | Third | 00 | 00 |
| 4. | Total % captured | 90 | 85 |
| | cells | | |
| 5. | Non-specific % | 10 | 15 |
| | capture | | |

### Example 20

Capture of Circulating Tumor Cells from Cancer Patients Blood.

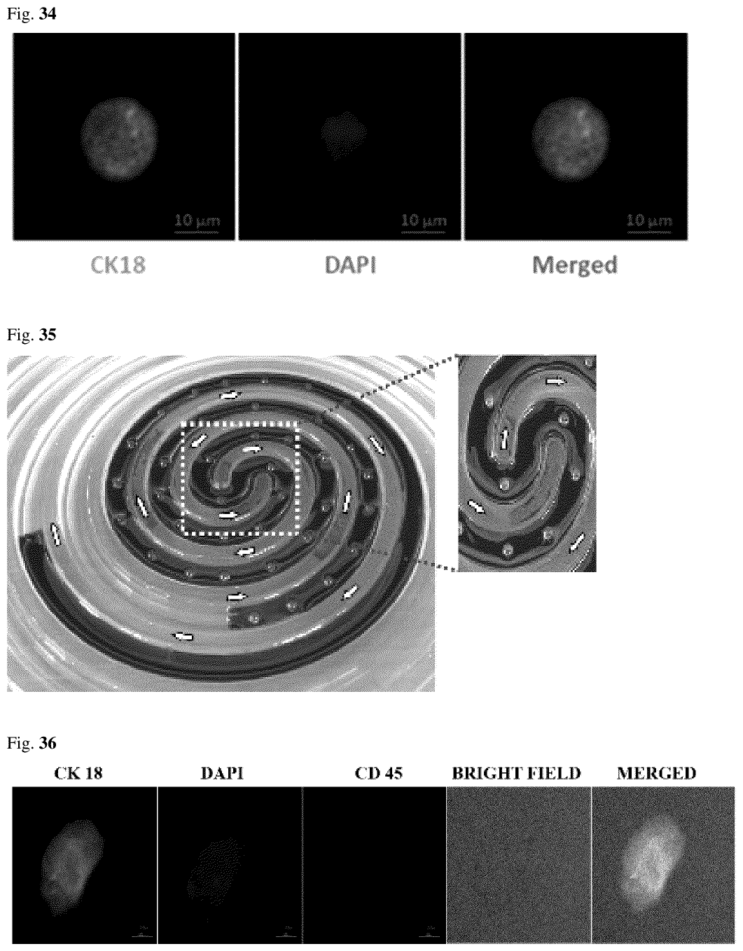

5 milliliters of cancer patients blood was incubated with mild agitation for 30 minutes on the spiral channel device 1, as described in example 1 (see FIG. 5) filled with glass beads of example 12 (FIG. 35). Circulating tumor cells captured were fixed for 5 minutes with absolute methanol, washed with phosphate buffered saline pH 7.4, and immuno-stained with anti-cytokeratin (CK-18) and anti-leucocyte common antigen (CD-45) antibodies for 1 hour followed by nuclear-staining probe 4′, 6-diamidino-2-phenylindole (DAPI) for 5 minutes.

Captured CTCs were observed and imaged under fluorescence scope (see FIG. 36).

### Example 21

Glass Beads Conjugated with Different Concentration of Antibody on Glass Beads.

600 milligrams (≃45 beads) GPTMS functionalised glass beads of example 11 of U.S. Pat. Appl. 17/069, 27711 were treated with different concentrations of anti-EpCAM namely, 0.75 micro gram, (Sample 21a) 1 microgram, (Sample 21b) and 1.25 micro gram (Sample 21c) (in 1 milliliter phosphate buffered saline pH 7.9 at 25° C. for 4 hours. The antibody conjugated glass beads were washed with 1 milliliter phosphate buffered saline pH, 7.4 three times and were stored in 1 milliliter phosphate buffered saline pH 7.4 at 4° C.

### Example 22

Transferrin Conjugated Glass Beads.

600 milligrams (≃45 beads) functionalised with glass beads of example 11 of U.S. patent application Ser. No. 17/069,277 were treated with 2.28 micro gram of transferrin in 50 mM of 1-Ethyl-3-(3-dimethyllaminopropyl)carbodiimide hydrochloride and 50 mM N-hydroxysuccinimide in 1 milliliter phosphate buffered saline pH 7.9, followed by rocking using rockymax at 25° C. for 4 hours. Thereafter, the transferrin conjugated glass beads were washed with 1 milliliter phosphate buffered saline, pH 7.4 three times and were stored in 1 milliliter phosphate buffered saline pH 7.4 at 4° C.

### Example 23

Glass Beads Conjugated with Anti-EpCAM and Transferrin.

600 milligrams (≃45 beads) GPTMS functionalised as described above when treated with 1 micro gram of anti-EpCAM and 2.28 micro gram transferrin (Molar ratio 1:1) in 50 mM of 1-Ethyl-3-(3-dimethyllaminopropyl)carbodiimide hydrochloride (EDC-HCl) and 50 mM N-hydroxysuccinimide (NHS) in 1 milliliter phosphate buffered saline pH 7.9, followed by rocking using rockymax at 25° C. for 4 hours. Thereafter, the conjugated glass beads were washed with 1 milliliter phosphate buffered saline pH 7.4 three times and stored in 1 milliliter phosphate buffered saline, pH 7.4 at 4° C.

### Example 24

Capture of Breast Cancer (MCF 7) Cells Using Glass Beads of Example 21.

The glass beads of example 21 were incubated with breast cancer cells and the cell suspension supernatant was discarded. The glass beads were washed 3× times with phosphate buffered saline, pH 7.4 to remove any unbound MCF 7 cells. The glass beads were treated with 1× Trypsin-EDTA solution for 5 minutes at 37° C. to detach the captured MCF7 cells. Cells detached from glass beads were fixed with 1 milliliter of chilled (4° C.) absolute methanol for 5 minutes, washed with phosphate buffered saline pH 7.4, and immuno-stained with anti-cytokeratin (CK-18) antibodies and with nuclear-staining probe 4′, 6-diamidino-2-phenylindole (DAPI). Captured cells were observed and imaged under fluorescence scope (FIG. 37). Number of cells captured and were accounted in Table 6.

TABLE 6
Capture of breast cancer cells MCF7 of glass beads of example 21.

| Sample | No. of captured MCF7 cells |
|---|---|
| 21 a | 304 |
| 21 b | 456 |
| 21 c | 532 |

### Example 25

Capture of Breast Cancer Cells (MCF7) on Conjugated Glass Beads Using Different Ligands.

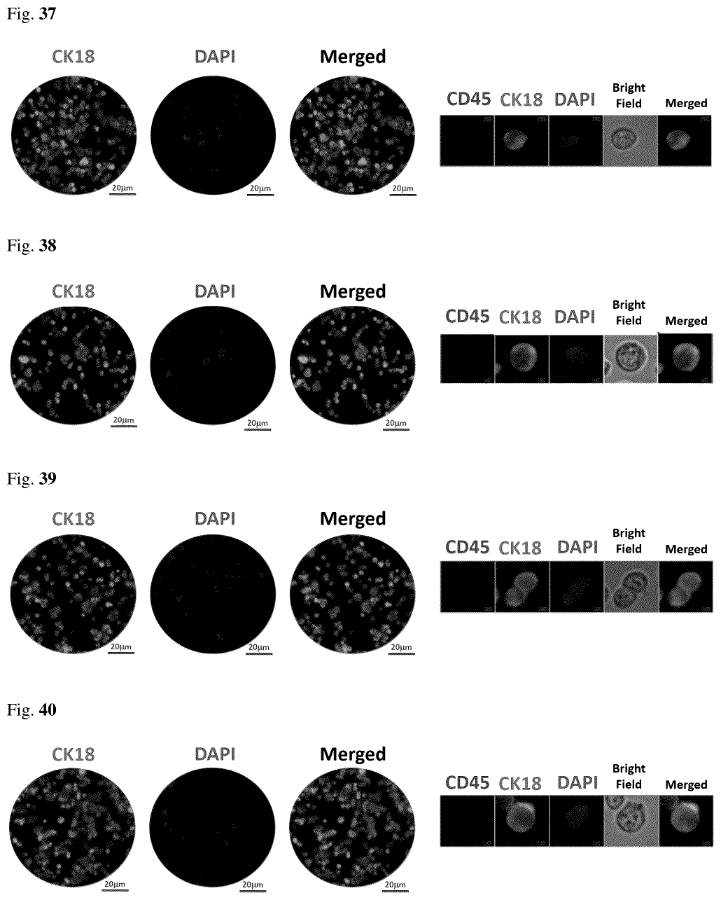

Glass beads of examples 21, 22, 23 were placed in spiral device of example 1 and incubated with 500 μl solution containing 4000 MCF7 cells/milliliter for 30 minutes. Following incubation with cells, the cell suspension supernatants were discarded, and glass beads were washed 3× times with phosphate buffered saline pH 7.4 to remove any unbound MCF7 cells. The glass beads were treated with 1× Trypsin-EDTA solution for 5 minutes at 37° C. to detach the captured MCF7 cells. Cells detached from the glass beads were fixed with 1 milliliter chilled absolute methanol for 5 minutes, washed with phosphate buffered saline pH 7.4, and immuno-stained with anti-cytokeratin (CK-18) antibodies and with nuclear-staining probe 4′, 6-diamidino-2-phenylindole (DAPI). Captured cells were observed and imaged under fluorescence scope (FIGS. 37-40). See Table 7.

TABLE 7
Capture of cancer cells using different
ligands (examples 21, 22 and 23)

| Example | No. of captured MCF-7 cells |
|---|---|
| Example 22 | 228 |
| Example 21 (Sample 21b) | 456 |
| Example 23 | 608 |

### Example 26

Effect of Incubation Time on Cell Capture.

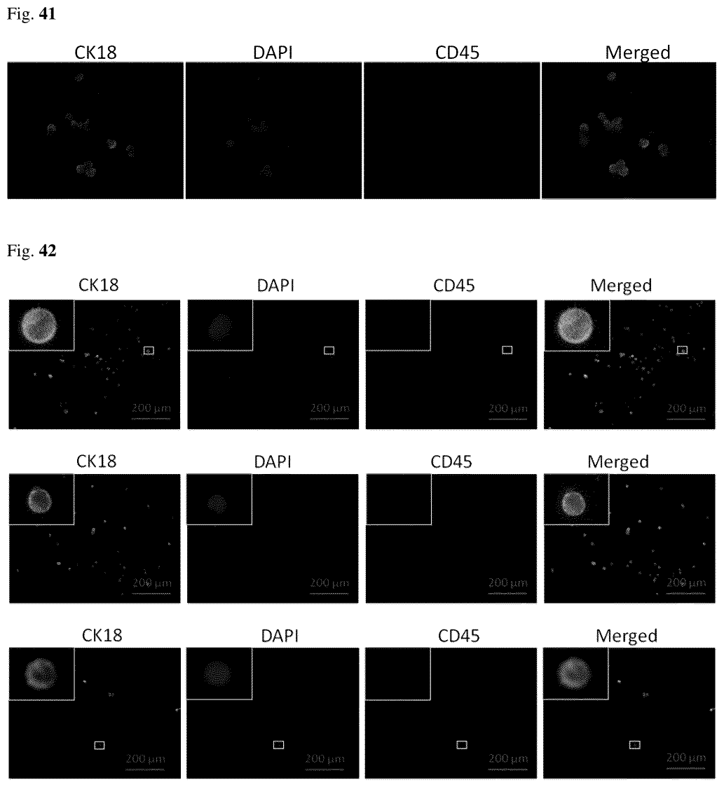

Glass beads of example 21 (sample 21 b) were filled in spiral channel device of example 1 and incubated for 2, 15, 30 minutes with 4000 MCF7 cells/milliliter in device. Following incubation, the cell suspension supernatants were discarded, and glass beads were washed 3× times, with phosphate buffered saline pH 7.4 to remove any unbound cells. The glass beads were treated with 1× Trypsin-EDTA solution for 5 minutes at 37° C. to detach the captured cells. Cells detached were fixed with 1 milliliter chilled absolute methanol (4° C.) for 5 minutes, washed with phosphate buffered saline pH 7.4, and immuno-stained with anti-cytokeratin (CK-18) antibodies and with nuclear-staining probe 4′, 6-diamidino-2-phenylindole (DAPI). Captured cells were observed and imaged under fluorescence scope (FIGS. 41 and 42). The effect of incubation time on the number of cell captured is summarized in Table 8.

TABLE 8
The effect of incubation time on the number of cells captured.

| Cell incubation time (min) | No. of captured MCF7 cells |
|---|---|
| 2 | 76 |
| 15 | 152 |
| 30 | 532 |

### Example 27

Cell Capturing Order Using Increasing Number of Beads.

Glass beads of example 21 (sample 21 b) 3, 6, 12 in numbers having 1.0 microgram anti-Epithelial Cell Anti-EpCAM and 12 beads glass beads of example 11 of U.S. patent application Ser. No. 17/069,277 were incubated with 4000 cells/milliliter MCF7 cells for 30 minutes in the spiral device example 1.

Following incubation, the cell suspension supernatants were discarded and the said glass beads were washed three times with phosphate buffered saline, pH 7.4 to remove any unbound cells. The glass beads were treated with 1× Trypsin-EDTA solution for 5 minutes at 37° C. to detach the captured cells. Cells detached from the glass beads were fixed with 1 milliliter of chilled absolute methanol (4° C.) for 5 minutes, washed with phosphate buffered saline pH 7.4, and immuno-stained with anti-cytokeratin (CK-18) antibodies and with nuclear-staining probe 4′, 6-diamidino-2-phenylindole (DAPI). Captured cells were observed and imaged under fluorescence scope. Cells captured were accounted and the results were summarized in Table 9.

TABLE 9
Cell capture ratios using increasing number of beads of example 12

| | MCF7 | No. of captured | |
|---|---|---|---|
| Number of glass beads | cells | cells/(Ratio) | |
| 3 | 4000 | 532 | (13.3%) |
| 6 | 8000 | 684 | (8.55%) |
| 12 | 16000 | 912 | (5.7%) |
| Total | 28000 | 2128 | (13.15%) |
| 12 (glass beads of example 11 | 4000 | 39 | (1%) |
| of U.S. Pat. Appl. 17/069,277 | | | |

| Number of glass beads | HeLa | No. of captured |
|---|---|---|
| of example 12 | cells | HeLa cells |
| 12 | 16000 | 952 (5.95%) |

### Example 28

Cell Capture Using the Increasing Number of Cells.

12 Glass beads of example 12 were incubated for 30 minutes with increasing number of MCF7 cells from 100-300 cells/milliliter in device example 1.

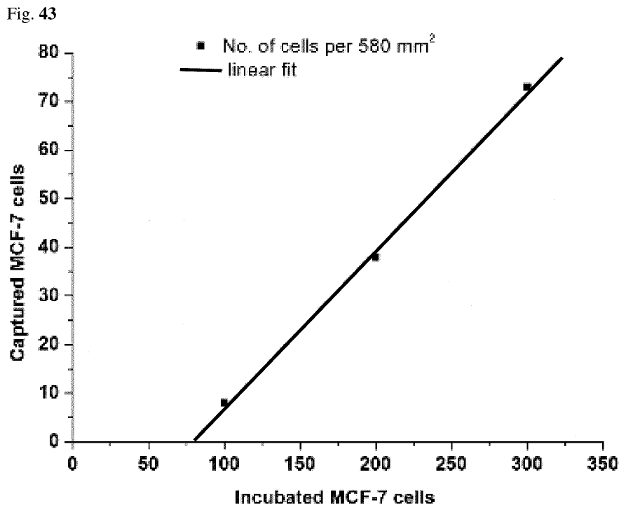

Following incubation, the cell suspension supernatants were discarded, and the glass beads were washed three times with phosphate buffered saline pH 7.4 to remove any unbound cells. The glass beads were treated with 1× Trypsin-EDTA solution for 5 minutes at 37° C. to detach the captured cells. Cells detached from the glass beads were fixed with 1 milliliter of chilled absolute methanol (4° C.) for 5 minutes, washed with phosphate buffered saline pH 7.4, and immuno-stained with anti-cytokeratin (CK-18) antibodies and with nuclear-staining probe 4′, 6-diamidino-2-phenylindole (DAPI). Captured cells were observed and imaged under fluorescence scope and the graph was plotted as shown in FIG. 43.

### Example 29

Cell Capture Using Glass Beads Synthesized Using Increasing Concentration of Antibody.

12 glass beads of example 21 (sample 21 a-c) were incubated for 30 minutes with 4000 cells/milliliter MCF7 cells in device example 1.

Following incubation, the cell suspension supernatants were discarded, and glass beads were washed three times with phosphate buffered saline pH 7.4 to remove any unbound cells. The glass beads were treated with 1× Trypsin-EDTA solution for 5 minutes at 37° C. to detach the captured cells. Cells detached from the glass beads were fixed with 1 milliliter of chilled absolute methanol for 5 minutes, washed with phosphate buffered saline pH 7.4, and immuno-stained with anti-cytokeratin (CK-18) antibodies and with nuclear-staining probe 4′, 6-diamidino-2-phenylindole (DAPI). The captured cells were observed and imaged under fluorescence scope Cells captured were accounted and plotted as a graph as shown in FIG. 44.

Example 30

% Hemolysis Assay for Different Devices.

Red Blood Cells (RBCs) from cancer patient's blood (5 milliliters) were separated by centrifugation (500×g, 5 minutes) at room temperature and washed in 150 mM sodium chloride (NaCl) solution two times, and resuspended in 5 milliliters phosphate-buffered saline, pH 7.4. The resuspended RBCs were incubated in the respective devices at different flow rates as represented in Table 10 at room temperature. Hemolysis was determined for devices packed using glass beads of example 12. 0.5 milliliter of blood exiting the device was collected and centrifuged at 500×g, 5 minutes, room temperature, and supernatants were collected and analyzed for hemolysis using UV spectroscopy at 540 nm. The percentage hemolysis was estimated against a negative control phosphate buffered saline with pH 7.4 and positive control 0.5% Triton-X100. The percentage of hemolysis is shown in Table 10.

TABLE 10
Percentage hemolysis of Red Blood Cells.

| | | Flow |
|---|---|---|
| Sr. | Column (% packing) | rate(milliliter/hr) |
| 1. | Positive Control (Phosphate buffered saline) | — |
| 2. | Negative Control (TritonX100 0.5%) | — |
| 3. | FIG. 5 | 0 |
| 4. | FIG. 5 | 100 |
| 5. | FIG. 5 | 50 |
| 6. | FIG. 5 | 1 |
| 7. | FIG. 5 | 0 |
| 8. | FIG. 5 | 100 |
| 9. | FIG. 5 | 50 |
| 10. | FIG. 5 | 1 |
| 15. | FIG. 23A (10) | 100 |
| 16. | FIG. 23A (10) | 50 |
| 17. | FIG. 23A (10) | 1 |
| 18. | FIG. 23A (30) | 100 |
| 19. | FIG. 23A (30) | 50 |
| 20. | FIG. 23A (30) | 1 |
| 21. | FIG. 23A (60) | 100 |
| 22. | FIG. 23A (60) | 50 |
| 23. | FIG. 23B (60) | 1 |
| 24. | FIG. 23A (90) | 100 |
| 25. | FIG. 23B (90) | 50 |
| 26. | FIG. 23A (90) | 1 |
| 27. | FIG. 23B (10) | 100 |
| 28. | FIG. 23B (10) | 50 |
| 29. | FIG. 23B (10) | 1 |
| 30. | FIG. 23B (30) | 100 |
| 31. | FIG. 23B (30) | 50 |
| 32. | FIG. 23B (30) | 1 |
| 33. | FIG. 23B (60) | 100 |
| 34. | FIG. 23B (60) | 50 |
| 35. | FIG. 23B (60) | 1 |
| 36. | FIG. 23B (90) | 100 |
| 37. | FIG. 23B (90) | 50 |
| 38. | FIG. 23B (90) | 1 |

The % hemolysis for positive control was 0%, for negative control was 90% and for all other examples was 0%. The columns Sr. Numbers 7-14, 21-32 were made from glass and Sr. numbers 3-6, 15-20 were made from silicone.

### Example 31

Breast Cancer Cells (MCF-7) Capture.

Breast cancer cells, MCF-7 were incubated for 15 minutes with different devices as enumerated in Table 3. The adsorbed cells were fixed with absolute ethanol and immunostained with antibodies against cytokeratin (CK-18) and leukocyte, a common antigen (CD-45) and with nuclear-staining probe 4′,6-diamidino-2-phenylindole (DAPI). Isolated cells were imaged and observed under fluorescence scope as in FIGS. 41 and 42.

The results of MCF 7 cell capture and CTC capture are summarized in Table 11.

TABLE 11
Capturing MCF-7 cells and CTCs.

| Sr. | Exam- | | Cell Type |
|---|---|---|---|
| No. | ple | Description | Captured |
| 1. | 13 | Hollow cylindrical glass column linked with | MCF-7 |
| | | anti-epithelial cell adhesion molecule | |
| | | antibody as a device chamber. | |
| 2. | 5 | Hollow glass column with reservoir linked | MCF-7 |
| | | with anti-epithelial cell adhesion molecule | |
| | | antibody as a device chamber. | |
| 3. | 13 | Hollow cylindrical glass column linked with | CTC |
| | | anti-epithelial cell adhesion molecule | |
| | | antibody as a device chamber. | |
| 4. | 5 | Hollow glass column with reservoir linked | CTC |
| | | with anti-epithelial cell adhesion molecule | |
| | | antibody as a chamber device. | |
| 5. | 13 | Hollow cylindrical glass column packed with | CTC |
| | | anti-epithelial cell adhesion molecule | |
| | | antibody linked glass beads of example 12 | |
| | | were with 10% packing volume as a device | |
| | | chamber. | |

### Example 32

Capture of CTCs Using Cancer Patient's Blood.

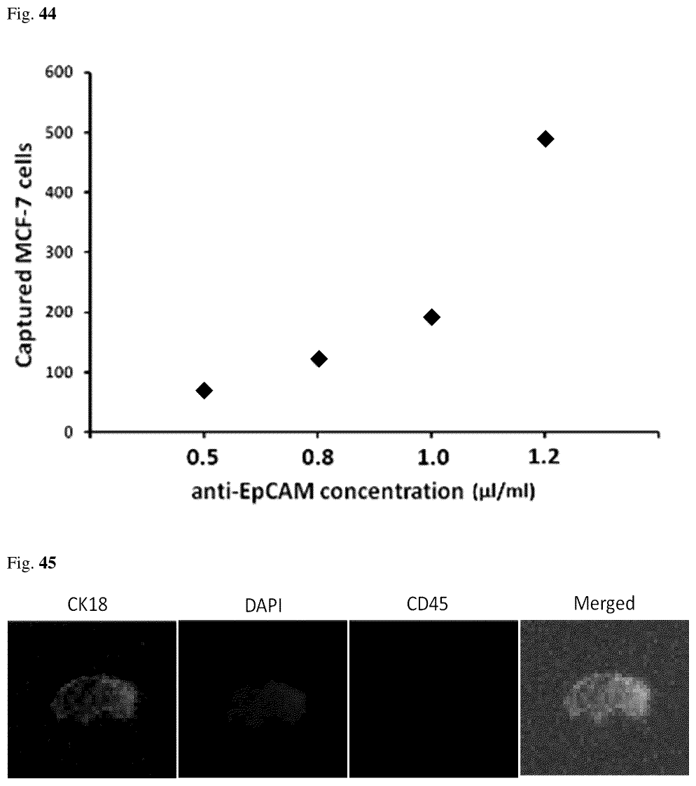

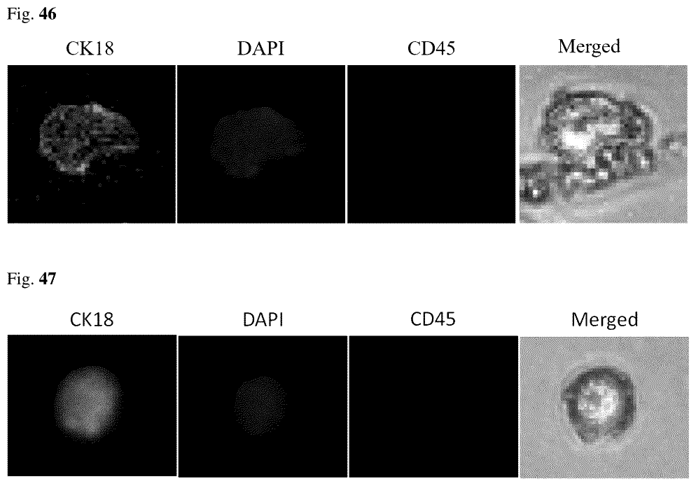

1.5 milliliters Cancer patient blood sample was incubated in device 1 containing glass beads of example 12 for 15 minutes. Captured CTCs were fixed with absolute ethanol for 5 minutes and immuno-stained with cytokeratin (CK-18), leukocyte common antigen (CD-45) and with nuclear-staining probe 4′,6-diamidino-2-phenylindole (DAPI). The captured cells were observed and imaged using fluorescence scope (see FIGS. 45 and 46).

### Example 33

Removal of Cells after Capture.

The cells that were captured as described in example 30 were detached using trypsinization method. Cells released were than imaged under the florescence scope (FIG. 47).

### Example 34

Destruction of Cells Using Anticancer Drug.

1.5 milliliters blood of a cancer patient was incubated with 300 grams of glass beads of example 12 for 5 minutes. Cell were fixed with absolute methanol and immuno-stained with cytokeratin (CK-18), leucocyte common antigen (CD-45) and with nuclear-staining probe 4′,6-diamidino-2-phenylindole (DAPI). Captured cells were imaged under fluorescence scope, wherein 3 CTCs were isolated and incubated for two hours with 30 micrograms of Cisplatin in 100 microliter phosphate buffered saline pH 7.4.

No CTCs were observed when attempt was made to fix the cells.

### Example 35

Animal Histopathological Outcome and Safety.

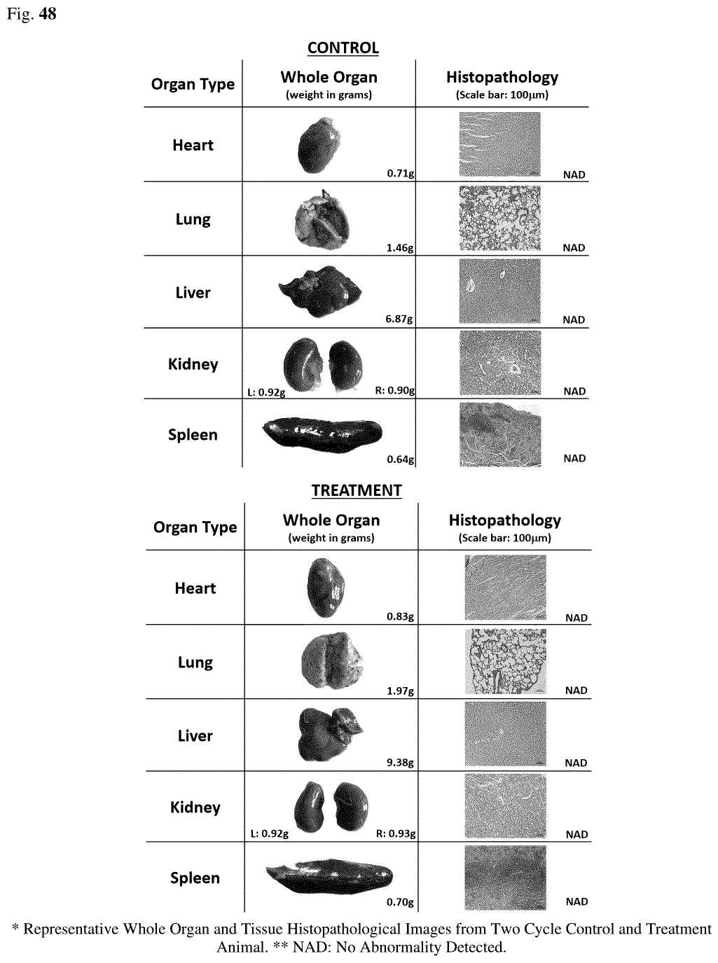

The glass beads of example 12 were evaluated using animal rat models (FIG. 48). 400 microlitres of rat blood was withdrawn and exposed to the said glass beads for 10 minutes and the blood was re-transfused to the animals. The animals were incubated for 14 days. Post this period the animals were sacrificed and whole organs like heart, lung, liver, kidney and spleen were evaluated for tissue pathology changes. It was noted that all animals survived and no tissue abnormality was observed in animals exposed with glass beads of example 12 and as in control group (FIG. 48).

### Example 36

Method of Treatment of a Cancer Patient

5 milliliters of cancer patient's blood was taken in 10 milliliters plastic syringe having a piston. The syringe and piston was placed and adjusted to a pump having adjustable pressures exerted to the piston for fluid flow. The pressure to the piston and the flow of the blood for the experiment to was adjustable from 1-20 seconds. The outlet of syringe was connected to channel device of example 1 consisting glass beads of example 12, using silicone tubing.

The technique of drawing blood from a cancer patient and returning to the venous system through a standard dual-lumen catheter or access port or a double needle system is well known. A peristaltic pump can maintain a desired flow conditions. The pump can provide constant flow and can maintain the desired pressure.

Similarly, the blood can be circulated into device by gravity flow (with a defined height for the holding blood bottles).

The device adsorbs the CTCs and other toxic components from blood circulated into the spiral channel device having variable number of glass beads. Following this the CTCs are detached from the glass beads, while the blood is returned to the patient.

All references cited herein are incorporated herein by reference in their entirety for all purposes.

1. A hemocompatible device for the capture and removal of a disease-causing agent from blood of a cancer patient, comprising a channel and an adsorbent that selectively binds to the disease-causing agent and allows the passage of the blood through the device preserving the morphology and viability of the red blood cells.

2. The hemocompatible device of claim 1, wherein the device for the capture and removal of the disease-causing agent is a spiral channel.

3. The hemocompatible device of claim 1, wherein the device comprises a hollow column.

4. The hemocompatible device of claim 1, wherein the device comprises a hollow column and a reservoir.

5. The hemocompatible device of claim 1, wherein the device comprises a spiral or coiled hollow column.

6. The hemocompatible device of claim 4, wherein the device comprises multiple hollow columns.

7. The hemocompatible device of claim 4, wherein the device comprises multiple hollow columns arranged in series.

8. The hemocompatible device of claim 4, wherein the device comprises multiple hollow columns arranged in parallel.

9. The hemocompatible device of claim 1, wherein the device is fabricated from a material selected from glass, steel, silicone, fluorinated polymers and a combination thereof.

10. An adsorbent that selectively binds to a disease-causing agent from the blood of a cancer patient comprising a substrate functionalized with a functionalizing agent that is linked to a ligand that selectively binds to the disease-causing agent.

11. The adsorbent of claim 10, wherein the functionalizing agent is selected from glutathione, cysteine, citric acid, (3-aminopropyl)triethoxysilane (APTES), (3-glycidyloxypropyl)trimethoxysilane (GPTMS), (3-glycidyloxypropyl) triethoxysilane (GPTES), (3-mercaptopropyl)trimethoxysilane (MPTMS), (3-mercaptopropyl) triethoxysilane (MPTES).

12. The adsorbent of claim 10, wherein the ligand is selected from anti-epithelial cell adhesion molecule antibody (anti EpCAM), a protein (transferrin, Bovine Serum Albumin (BSA)), and a carbohydrate (N-acetyl glucosamine) (NAG).

13. The hemocompatible device of claim 1, wherein an internal surface of the device is the adsorbent functionalized and linked to a ligand that selectively binds to the disease-causing agent, and wherein the disease-causing agent is a circulating cancer cell.

14. The hemocompatible device of claim 13, wherein the device comprises more than one type of ligand that selectively binds to the circulating cancer cell.

15. The hemocompatible device of claim 13, wherein the adsorbent is glass beads that are functionalized and linked to a ligand that selectively binds to the circulating cancer cell.

16. The hemocompatible device of claim 15, wherein the adsorbent glass beads are packed into the device, and the packed volume is in the range of from about 10% to about 90% v/v.

17. The hemocompatible device of claim 1, wherein the disease-causing agent is a circulating cancer cell selected from a breast cancer cell, a prostate cancer cell, a colorectal cancer cell, a lung cancer cell, a pancreatic cancer cell, an ovarian cancer cell, a bladder cancer cell, an endometrine or uterine cancer cell, a cervical cancer cell, a liver cancer cell, a renal cancer cell, a thyroid cancer cell, a bone cancer cell, a lymphoma cell, a melanoma cell and a non-melanoma skin cancer cell.

18. A method for capturing and removing a disease-causing agent from blood of a cancer patient comprising passing the blood of a cancer patient through a hemocompatible device comprising a channel and an adsorbent that selectively binds to the disease-causing agent and allows the passage of the blood of the cancer patient through the device, wherein the device preserves the morphology and viability of the cancer patient's red blood cells.

19. The method of claim 18, wherein the flow of blood through the device is in the laminar region.

20. The method of claim 18, wherein the blood is passed through the device over a period of from about 5 minutes to about 60 minutes.

21. A method for destroying a disease-causing agent from the blood of a cancer patient comprising passing the blood of a cancer patient through a hemocompatible device of claim 1, and, incubating an adsorbate captured in the hemocompatible device with an anticancer drug selected from Vancomycin, Metformin, Doxorubicin, Methotrexate, Paclitaxel, 5-Fluorouracil, Cisplatin, and Camptothecin, Docetaxel, Oxaliplatin, and Cyclophosphamide, wherein the adsorbate is a circulating cancer cell.

22. A method for destroying a disease-causing agent from the blood of a cancer patient comprising passing the blood of a cancer patient through a hemocompatible device of claim 1, and incubating the cancer cells captured in the hemocompatible device with at least one anticancer drug selected from Vancomycin, Metformin, Doxorubicin, Methotrexate, Paclitaxel, 5-Fluorouracil, Cisplatin, and Camptothecin, Docetaxel, Oxaliplatin, and Cyclophosphamide or a combination thereof.

23. A method of treating a cancer patient, comprising the steps of a) removing a fixed amount of blood from the cancer patient, b) passing the blood through a hemocompatible device of claim 1 for capturing a disease-causing agent, and c) infusing the treated blood back in to the cancer patient, wherein the method is effective for removing cancer cells from the blood of the patient but preserves the morphology and viability of the cancer patient's red blood cells.
